(19) United States
(12) Patent Application Publication
(10) Pub. No.: US 2023/0025218 A1
(43) Pub. Date: Jan. 26, 2023

(54) TOILET SYSTEM WITH SENSORS FOR MEASURING EXCRETION OUTPUT OF A USER

(71) Applicant: Measurelet ApS, Hørsholm (DK)

(72) Inventors: Marie Lommer BAGGER, Virum (DK); Morten BO SØNDERGAARD SVENDSEN, Kongens Lyngby (DK)

(73) Assignee: Measurelet ApS, Hørsholm (DK)

(21) Appl. No.: 17/816,213

(22) Filed: Jul. 29, 2022

Related U.S. Application Data
(63) Continuation of application No. PCT/DK2021/050029, filed on Jan. 29, 2021.

(30) Foreign Application Priority Data
Jan. 31, 2020 (DK) 202070063

(51) Int. Cl. A61B 10/00; A61B 5/20; A61B 5/00; E03D 11/00; A61B 7/00; G01G 19/52

(52) U.S. Cl. CPC A61B 10/0038; A61B 5/208; A61B 5/4283; E03D 11/00; A61B 7/008; A61B 5/486; G01G 19/52; A61B 5/7435; A61B 2562/0204; A61B 2562/0247

(57) ABSTRACT

A toilet system comprising a bowl and an outlet, the toilet system being adapted for discerning between types of excretion excreted by a user of the toilet system, where the toilet system comprises at least one first sensor device configured to capture at least one signal indicative of a type of excretion excreted by a user of the toilet system, and at least one second sensor device configured to capture at least one signal indicative of a quantity of excretion excreted by a user of the toilet system.

### CROSS-REFERENCE TO RELATED APPLICATIONS

The present application is a continuation of International Application No. PCT/DK2021/050029, filed on Jan. 29, 2021, which claims priority to Denmark Application No. 202070063, filed Jan. 31, 2020, entitled “A TOILET SYSTEM WITH SENSORS FOR MEASURING EXCRETION OUTPUT OF A USER, A FLUID BALANCE MONITORING SYSTEM AND A METHOD FOR DETERMINING AND MONITORING THE FLUID BALANCE OF A SUBJECT”. The International Application, having No. PCT/DK2021/050029, is herein incorporated by reference in its entirety.

### TECHNICAL FIELD

The present invention relates to a toilet system comprising a bowl and an outlet, the toilet system being adapted for discerning between types of excretion excreted into the bowl by a user of the toilet system.

### BACKGROUND ART

Knowledge of the type and quantity of excretion excreted by patients is of great importance within the health sector since healthcare professionals, such as nurses and doctors, use this information for diagnostic purposes and for monitoring the progress of diseases and convalescence in patients. For instance, this information may be of great value in determining the correct treatment for a patient and whether the treatment chosen is effective or needs adjustment.

Toilets adapted for measuring faecal and urinal output of a user are commonly known. However, by far most known solutions are limited to addressing the issue of measuring the quantity of urine excreted into a toilet by a user. This has in the prior art been done both using weight sensors and using pressure sensors.

Within the health sector, particularly on hospitals, it is therefore furthermore known as common practice for healthcare professionals to measure the quantity of feces excreted into a toilet or a bedpan by a patient by physically collecting and weighing the feces. This process is, however, extremely time consuming and cumbersome, and furthermore impractical from a hygiene-related perspective, for users and healthcare professionals alike. Also, the use of bedpans may be unappealing or even humiliating to the patient or user.

Particularly, it is desired to provide an improved fluid balance monitoring system and an improved method for determining and monitoring the fluid balance of a subject.

### SUMMARY OF INVENTION

The following presents a simplified summary of the disclosure in order to provide a basic understanding of certain embodiments of the invention. This summary is not an extensive overview of the disclosure, and it does not identify key/critical elements of the invention or delineate the scope of the invention. Its sole purpose is to present some concepts disclosed herein in a simplified form as a prelude to the more detailed description that is presented later.

It would be beneficial to provide a toilet with which the precision of the measurements performed is increased, which is capable of properly discerning between faecal and urinal matter when excreted into the bowl of the toilet by a user, and which is able to accurately assess the quantity of faecal matter when excreted into the bowl of the toilet by a user. It would also be beneficial to provide a toilet which is easy and quick to use, and which provides for a more appealing and hygienic solution, for users, patients, and healthcare professionals alike.

In one embodiment, a toilet system comprising a bowl and an outlet is disclosed, and the toilet system is adapted for discerning between types of excretion excreted into the bowl by a user of the toilet system. In one aspect, the toilet system further comprises at least one first sensor device configured to capture at least one signal indicative of a type of excretion excreted into the bowl by a user of the toilet system and at least one second sensor device configured to capture at least one signal indicative of a quantity of excretion excreted into the bowl by a user of the toilet system.

By providing at least one first sensor device configured to capture at least one signal indicative of a type of excretion excreted into the bowl by a user of the toilet system, a toilet system is provided which is capable of properly discerning between faecal and urinal matter when excreted into the bowl of the toilet by a user or patient. By further providing at least one second sensor device configured to capture at least one signal indicative of a quantity of excretion excreted into the bowl by a user of the toilet system, a toilet system is provided which is also capable of assessing the quantity of faecal matter when excreted into the bowl of the toilet by a user or patient.

As the sensor devices capture the respective signals automatically and without the need of human intervention, a toilet system is provided which is furthermore easy and quick to use, particularly for the user or patient, and which provides for a more appealing and hygienic solution, for users, patients and healthcare professionals alike. Also, time and resources are saved. Furthermore, such a toilet system provides the data needed by healthcare professionals for diagnostic purposes and for monitoring the progress of diseases and convalescence in patients in a simple, hygienic and reliable manner.

In an embodiment, the at least one first sensor device is configured to measure sounds. The inventors have found that by measuring the sound related to a user excreting an excretion, it becomes possible with a particularly high degree of certainty and precision to discern between faecal and urinal matter when excreted into the bowl of a toilet.

Furthermore, the inventors have found that by using sound measurements it even becomes possible to also identify the excretion or disposal of other types of matter, such as flatulence or paper, into the bowl of the toilet system from the excretion of faecal and urinal matter, and thereby to remove unwanted and/or irrelevant data such as false positives from the resulting data.

Thereby a toilet system is provided with which the precision of the measurements performed is increased, and which is capable of properly and with a high precision discerning between faecal and urinal matter when excreted into the bowl of the toilet by a patient or user. Additionally, such a type of sensor need not come into physical contact with the excretion in order to capture the needed measurements and signals. Thereby, the hygiene and durability of the toilet system is further increased as it is easy to clean and keep clean.

In an embodiment, the at least one first sensor device comprises a vibration sensor, such as an acoustic sensor or a microphone. Thereby a toilet system is provided which is particularly simple in construction, and which is particularly easy to clean.

In an embodiment, the at least one first sensor device is arranged in or at the toilet seat. Thereby a toilet system is provided which the captured signals indicative of a type of excretion excreted into the bowl by a user of the toilet system is provided with a high degree of detail. This especially applies to such signals in the form of sound signals, and especially such sound signals produced by the patient or user.

In an embodiment the at least one first sensor device is arranged in or at the bowl above a waterline. Thereby a toilet system is provided which the capture of signals indicative of a type of excretion excreted into the bowl by a user of the toilet system is optimized and provided with a particularly great detail. The inventors have shown that due to the acoustics prevailing in the bowl above the water line, this especially applies to such signals in the form of sound signals, and especially such sound signals produced by the patient or user and/or such sound signals produced when excretions come into contact with the bowl or materials in the bowl.

In an embodiment, the at least one first sensor device is arranged in or at the bowl below the waterline. Thereby a toilet system is provided which the capture of signals indicative of a type of excretion excreted into the bowl by a user of the toilet system is optimized and provided with a particularly great detail. The inventors have shown that due to the acoustics prevailing in the bowl below the water line, this especially applies to such signals in the form of sound signals, and especially such sound signals produced by the excretions upon impact with the water in the bowl.

In an embodiment, the at least one first sensor device further comprises any one or more of a pressure sensor, a radar, an image capturing device, a capacitive sensor, and flow rate sensor. Thereby, further data for determining the type of excretion excreted into a bowl of a toilet system is provided in a particularly simple, straight forward and cost-efficient manner. This in turn provides for a determination of the type of excretion being particularly accurate. Capacitive sensors can be mounted on the outside of the water ways, water trap and the like of the toilet system, such as even outside the bowl of the toilet system, whereby a further improvement of the hygiene is achieved.

In an embodiment, the at least one first sensor device is configured to transmit the at least one signal to a data analysis device. In an embodiment, the at least one second sensor device is configured to transmit the at least one signal to a data analysis device. As the first sensor device and/or the second sensor device thus also transmits the captured signals to the analysis device automatically and without the need of human intervention, a toilet system is provided which is particularly easy and quick to use, particularly for the user or patient, and which provides for a more appealing and hygienic solution, for users, patients and healthcare professionals alike. Furthermore, such a toilet system provides the data needed by healthcare professionals for diagnostic purposes and for monitoring the progress of diseases and convalescence in patients in a particularly simple, hygienic and reliable manner, saving both time and resources for the involved individuals.

In an embodiment, the at least one second sensor device is configured to measure a volume of liquid indicative of a quantity of excretion excreted into the bowl by a user of the toilet system. Thereby a toilet system is provided which is able to accurately assess the quantity of faecal or urinal matter when excreted into the bowl of the toilet by a user, and with which the precision and degree of detail of the data needed by healthcare professionals for diagnostic purposes and for monitoring the progress of diseases and convalescence in patients is increased.

In an embodiment, the at least one second sensor device is arranged in the outlet of the toilet system in a position downstream of a water seal arranged between the bowl and the outlet of the toilet system. Such a positioning of the second sensor device can be advantageous especially in toilets with a water seal, a water trap or a u-bend in the outlet as the normal water level in the bowl of such toilets is flush with the upper limit defined by the water seal. Therefore, even a small amount of excretion deposited in the bowl will cause the water in the water seal to spill over into the outlet downstream of the water seal, such that the amount of spilled over water may easily be measured by a second sensor device positioned downstream of the water seal.

The at least one second sensor device may be any one or more of a weight sensor, a level sensor and a flow sensor. Thereby a toilet system is provided which is capable of properly and with a high precision capturing a signal indicative of the quantity of faecal or urinal matter excreted into the bowl of the toilet by a patient or user, and with which the precision of the measurements performed is increased.

In an embodiment, the toilet system further comprises a data analysis device, which comprises a data processing unit and a data storage unit. In this aspect, the data processing unit is configured to receive one or more signals transmitted by the at least one first sensor device and analyse the received one or more signals such as to produce a data output indicative of at least the type of excretion excreted into the bowl by a user of the toilet system. Thereby a toilet system is provided with which the precision of the measurements performed is increased, and which is capable of automatically and with a high precision discerning between faecal and urinal matter when excreted into the bowl of the toilet by a patient or user. Analysis of the data may thus be performed automatically and without any need for physical contact with the excretion in order to determine type. Thereby, time and costs otherwise spent by healthcare personnel, not only on analysis of the excretion, but also on associated sterilization and cleaning, is eliminated and the hygiene and durability of the toilet system is further increased. The analysis may be a comparison with existing data, e.g. from a database, and/or a statistical analysis. The analysis may for instance be performed in a neural network, a principal component analysis (PCA), a decision tree or a clustering. In an embodiment, the data analysis device is further configured to receive one or more signals transmitted by the at least one second sensor device, and analyse, using the data processing unit, the received one or more signals such as to produce a data output indicative of the quantity of excretion excreted into the bowl by a user of the toilet system. Thereby a toilet system is provided with which the above-mentioned advantages related to the data analysis device are also extended to the determination of the quantity of excretion.

In an embodiment, the data analysis device further comprises a data visualization unit, and the data processing unit is further configured to visualize on the data visualization unit the data output indicative of one or more of the type of excretion excreted into the bowl by a user of the toilet system and the quantity of excretion excreted into the bowl by a user of the toilet system. Thereby, a user or a healthcare professional may in a particularly simple and easy to understand manner be aided in interpreting the data obtained by the sensor devices and the result obtained by the data analysis device. Visualization of the data may thus be performed automatically and without any need for physical contact with the excretion or the toilet system. Thereby, time and costs otherwise spent by healthcare personal, not only on analysis and visualization, but also on associated sterilization and cleaning, is eliminated and the hygiene and durability of the toilet system is further increased.

In an embodiment, the toilet system further comprises an actuator configured to allow a user to indicate a type of excretion excreted into the bowl by the user of the toilet system and to transmit a signal indicative of the user's indication to an analysis device. Thereby, further data for determining the type of excretion excreted into a bowl of a toilet system is provided in a particularly simple, straight forward and cost efficient manner.

In another embodiment, the toilet system further comprises an actuator configured to allow a user to activate one or more of the toilet system and the analysis device before use of the toilet system. Such an actuator may for instance be a press button or an actuator, such as a motion sensor, configured to automatically register a user being in a vicinity of or touching the toilet system and activate one or more of the toilet system and the analysis device in reaction to registering a user touching the toilet system or a sound sensor configured to register a sound produced by a user locking the room or toilet stall. Thereby it becomes possible to allow the toilet system and/or the analysis device to enter into an active state only when the toilet system is to be used by a user, and otherwise be in a passive or inactive state, thereby saving power and costs.

In an embodiment, the toilet system further comprises a weight sensor arranged and configured to capture signals indicative of the user's weight before and after excretion, respectively. Thereby, further data for determining the quantity of excretion excreted into a bowl of a toilet system is provided in a simple and cost efficient manner.

In an embodiment, the first sensor device is an image capturing device arranged in or at any one of the toilet seat and the bowl above a waterline, and the second sensor device is arranged in the outlet of the toilet system in a position downstream of a water seal arranged between the bowl and the outlet of the toilet and comprises a chamber adapted for collecting liquid forced through the water seal and a weight sensor arranged and adapted for measuring the weight of the chamber and liquid contained in the chamber.

In an embodiment, the first sensor device is arranged such as to point downwards at an angle α of between 70 and 80 degrees, such as an angle α of 75 degrees, with the horizontal H or in an angle β of between 20 and 10 degrees, such as an angle β of 15 degrees, with the vertical V, where an angle of 90 degrees in this context corresponds to the vertical V or the direction of gravity.

In an embodiment, the first sensor device is located outside the central axis A of the seat and is rotated around the direction of gravity or vertical V to point in an angle γ of between 25 and 25 degrees, such as an angle γ of 30 degrees, with the central axis A of the seat.

The arrangement of the first sensor device above the waterline in the bowl as well as the above-described angles of the first sensor device each provide for an improved positioning and alignment of the first sensor device in order to obtain the best possible data for ensuring a reliable type recognition. In unison they serve for an optimized positioning and alignment of the first sensor device in order to obtain the best possible data for ensuring a reliable type recognition.

In another aspect, the invention pertains to a method for determining a type of excretion excreted into a bowl of a toilet system according to any one of the above claims by a user of the toilet system, and the method comprises the steps of (a) using at least one first sensor device of the toilet system, capturing a signal indicative of a type of excretion excreted into the bowl by a user of the toilet system, (b) transmitting the captured signal indicative of a type of excretion excreted into the bowl by a user of the toilet system to an analysis device comprising a data processing unit and a data storage unit, (c) receiving, by means of the data processing unit, one or more signals transmitted by the at least one first sensor device, and (d) analysing, by means of the data processing unit, the received one or more signals transmitted by the at least one the first sensor device such as to produce a data output indicative of the type of excretion excreted into the bowl by a user of the toilet system.

In an embodiment, the step of analysing comprises performing a comparison with existing data, e.g. from a database, and/or a statistical analysis. The analysis may for instance be performed in a neural network, a principal component analysis (PCA), a decision tree or a clustering. In an alternative embodiment, the method comprises the further steps of (a) using at least one second sensor device of the toilet system, capturing a signal indicative of a quantity of excretion excreted into the bowl by a user of the toilet system, (b) transmitting the captured signal indicative of a quantity of excretion excreted into the bowl by a user of the toilet system to the analysis device, (c) receiving, by means of the data processing unit, one or more signals transmitted by the at least one the second sensor device, and (d) analysing, by means of the data processing unit, the received one or more signals transmitted by the at least one the second sensor device such as to produce a data output indicative of the quantity of excretion excreted into the bowl by a user of the toilet system.

In an embodiment of the method, the data analysis device further comprises a data visualization unit, and the method further comprises the step of visualizing on the data visualization unit, using the data processing unit, the data output indicative of the type of excretion excreted into the bowl by a user of the toilet system and/or the data output indicative of the quantity of excretion excreted into the bowl by a user of the toilet system.

To avoid repetition, it is here noted that FIG. 12 schematically illustrates the steps of a method according to the second aspect of the invention. It is further noted that the four steps relating to the second sensor device and shown in the upper righthand side of FIG. 12 are optional steps in line with the embodiments of a method according to the second aspect of the invention as described above. For further details, reference is made to the Example enclosed in the detailed description following below.

In further aspects, a toilet system comprises a bowl and an outlet, and the toilet system is adapted for discerning between types of excretion excreted into the bowl by a user of the toilet system. In one aspect, the toilet system further includes at least one first sensor device configured to capture at least one signal indicative of a type of excretion excreted into the bowl by a user of the toilet system. In another aspect, the toilet system further comprises at least one first sensor device configured to capture at least one signal indicative of a type of excretion excreted into the bowl by a user of the toilet system, and the at least one first sensor device is configured to measure sounds. In a further aspect, the at least one first sensor device may further be configured to transmit the at least one signal to a data analysis device.

In another aspect, the toilet system further comprises at least one first sensor device configured to capture at least one signal indicative of a type of excretion excreted into the bowl by a user of the toilet system and to transmit the at least one signal to a data analysis device, and a data analysis device. In one aspect, the data analysis device comprises a data processing unit that is configured to (i) receive one or more signals transmitted by the at least one first sensor device, and (ii) analyse the received one or more signals such as to produce a data output indicative of at least the type of excretion excreted into the bowl by a user of the toilet system.

In certain embodiments, a toilet system is provided with which the quantity of faecal matter, when excreted into the bowl of the toilet system by a user, may be assessed in manner that is simple, reliable and precise, also over time. In certain embodiments, a toilet system is provided with which the sensor device may also be retrofitted to an existing toilet system, already being installed, in a simple and straight forward manner. In an alternative embodiment, a toilet system is provided, and the toilet comprises a bowl and an outlet. In one aspect, the toilet system is adapted for discerning between at least types of excretion excreted into the bowl by a user of the toilet system, and the toilet system further comprises at least one sensor device configured to capture at least one signal indicative of a quantity of excretion excreted into the bowl by a user of the toilet system. In a further aspect, the sensor device comprises a chamber provided on the outlet of the toilet system in a position downstream of a water seal arranged between the bowl and the outlet of the toilet system, and the chamber is arranged adjacent to at least a part of a lower half of the outlet pipe of the toilet system and is adapted to receive a quantity of liquid pressed through a water seal of the toilet system, and at least one through opening provided in the outlet of the toilet system in the position downstream of the water seal at which the chamber is provided such as to allow liquid being forced through the water seal to flow into the chamber, where the at least one through opening is arranged with a main axis extending perpendicular to a longitudinal axis L of the outlet pipe, and where the at least one through opening comprises a shape tapering in an upstream direction.

Thereby, and especially by providing at least one through opening in the outlet pipe, where the at least one through opening is arranged with a main axis extending perpendicular to a longitudinal axis of the outlet pipe and comprise a shape tapering in an upstream direction, a toilet system is provided with which the quantity of faecal matter, when excreted into the bowl of the toilet system by a user, may be assessed in a manner being simple, reliable and precise, also over time especially since the risk of clogging of the plurality of through openings is minimized.

With such a toilet system, the sensor device may also be retrofitted to an existing toilet system already being in use in a simple and straight forward manner by simply providing the chamber on and the at least one through opening in the existing outlet pipe.

Providing the at least one through opening arranged with a main axis extending perpendicular to a longitudinal axis L of the outlet pipe can allow the size of the openings, combined with the total flowrate, or pressure, in the outlet pipe, to then be used to determine how much liquid is captured in the chamber for measuring. It is noted that the quantity of liquid pressed through a water seal of the toilet system may for instance be between 5 ml/min and 1000 ml/min.

In an embodiment, a plurality of through openings are provided and the plurality of through openings comprise a largest size A measured in a direction perpendicular to the longitudinal axis L of the outlet and a largest size B measured in a direction parallel with the longitudinal axis L of the outlet, where neighboring through openings of the plurality of through openings are arranged spaced apart with a shortest distance C measured in a direction perpendicular to the longitudinal axis L of the outlet and a shortest distance D measured in a direction parallel with the longitudinal axis L of the outlet, and where the plurality of through openings are arranged in a pattern fulfilling the relations C<A and D<2B.

Small openings may clog if particulate matter is latched in them, eventually obstructing the flow that were intended for measuring. Also, in case of very small excretions which in turn force very small amount of liquid through the water seal, liquid intended for measuring may flow around the openings if the spacing between the openings is too large. The inventors have shown that by arranging a plurality of through openings in a pattern fulfilling the above-mentioned relations these two disadvantages may be overcome. This in turn further enhances the precision and reliability of the measurements performed.

In an embodiment, the at least one through opening comprises a shape tapering and ending in a tip end pointing in an upstream direction. Thereby, particulate matter is more easily and reliably guided past the openings, whereby the risk of the openings clogging if particulate matter is latched in them, eventually obstructing the flow that were intended for measuring, is lowered considerably or even avoided altogether.

In an embodiment, the at least one through opening comprises a cross sectional shape being polygonal, ellipsoid, circular, with any one or more of regular, concave, convex or irregular edge(s), or any combination thereof. Examples of polygonal shapes are for instance triangular, rectangular or diamond-shaped. Thus, openings in the form of slits are also feasible. Openings with such shapes have the advantage of obtaining the above-mentioned advantages while keeping the geometrical shape of the openings simple and easy to manufacture.

In an embodiment, the at least one through opening comprise a largest size A measured in a direction perpendicular to the longitudinal axis L of the outlet and a largest size B measured in a direction parallel with the longitudinal axis L of the outlet, and the size A is between 0.5 mm and 100 mm. Alternatively, or additionally, the size B is between 0.5 mm and 100 mm.

Small openings may clog too easily. Large openings, on the other hand, may result in too much particulate matter ending up in the chamber. This in turn may not only clog the chamber but may also render the measurements faulty or even useless due to the contribution to or influence on the measurements from or by the particulate matter stuck in the chamber and may render it more difficult to empty the chamber after measurements have been finished. The inventors have shown that by providing the at least one opening with a size within the above-mentioned intervals these two disadvantages, and especially the latter disadvantage, may be overcome. This in turn further enhances the precision and reliability of the measurements performed.

In an embodiment, the plurality of through openings comprise at least five openings. Thereby a sufficient number of openings is provided to ensure that a sufficient amount of liquid is captured in the chamber, which in turn ensures reliable and precise measurements.

In an embodiment, the sensor device further comprises a screening device arranged upstream of the at least one through opening or extending over the at least one through opening. Such a screening device has the advantage of contributing to leading particulate matter past the openings such as to avoid that too much particulate matter ends up in the chamber, while still allowing liquid to flow through the opening(s) and into the chamber. This in turn keeps the chamber from clogging and enables avoiding the measurements becoming faulty or even useless due to the contribution from the particulate matter stuck in the chamber.

In an embodiment, the sensor device further comprises a mass sensor or a weight sensor arranged and configured to monitor a weight of the chamber and to capture at least one signal indicative of a quantity of excretion excreted into the bowl by a user of the toilet system. Alternatively, or additionally, the sensor device may comprise a flow sensor arranged and configured to monitor a flow of liquid through the chamber and to capture at least one signal indicative of a quantity of excretion excreted into the bowl by a user of the toilet system. Thereby, a toilet system is provided with which signals indicative of a quantity of excretion excreted into the bowl by a user of the toilet system may be captured and transmitted to analysis in an analysis device in a particularly simple manner.

In an embodiment, the sensor device further comprises a mounting element adapted for enabling mounting the sensor device on an outlet pipe of a toilet system. Thereby, a toilet system is provided with which the sensor device may be retrofitted to an existing toilet system already being in use in a particularly simple and straight forward manner by simply using the mounting element when mounting the chamber on an existing outlet pipe.

The mounting element may be an element configured for being attached around an outlet pipe. The mounting element may also be a fastener such as a welding or a gluing.

In an embodiment, the chamber further comprises any one of an outlet and a pumping device adapted for pumping the contents of the chamber into the outlet of a toilet system. Thereby, the chamber may be emptied in a particularly simple and efficient manner. The contents of the chamber may for instance simply be pressed back through the at least one through opening and back into the outlet pipe.

In an embodiment, the toilet system may further comprise a movable device adapted for covering the at least one through opening, optionally from below, i.e. on the side facing the chamber, and a device for controlling the movable device and the flushing system of the toilet system in such a manner that flushing is delayed until the movable device is brought to cover the at least one through opening.

In an embodiment, the sensor device further comprises a valve mounted in connection with at least one of the one or more openings and configured to control inflow of liquid into the chamber. The valve may comprise an actuator. Such a valve has the advantage of contributing to leading particulate matter past the openings such as to avoid that too much particulate matter ends up in the chamber, while still allowing liquid to flow through the opening(s) and into the chamber. Such a valve may furthermore be used to accurately control the size of particulate matter allowed to flow past it into the chamber. This in turn keeps the chamber from clogging and enables avoiding the measurements becoming faulty or even useless due to the contribution from the particulate matter stuck in the chamber.

In an embodiment, especially an embodiment where the sensor device comprises a valve, the sensor device comprises only one opening. In an embodiment, the sensor device is further configured to transmit the at least one signal indicative of a quantity of excretion excreted into the bowl by a user of the toilet system to a data analysis device. In an embodiment, the toilet system further comprises an image sensor.

In another embodiment the invention pertains to a method of providing a toilet system with at least one sensor device configured to capture at least one signal indicative of a quantity of excretion excreted into the bowl by a user of the toilet system, and the method comprises the steps of (i) providing at least one through opening in at least a part of a lower half of the outlet of the toilet system in a position downstream of a water seal arranged between the bowl and the outlet of the toilet system such as to allow liquid being forced through the water seal to flow through the openings and into the chamber, where the at least one through opening is arranged with a main axis extending perpendicular to a longitudinal axis L of the outlet pipe, and where the at least one through opening comprises a shape including a tip end pointing in an upstream direction, (ii) providing a chamber adapted to receive a quantity of liquid pressed through a water seal of the toilet system, and (iii) arranging the chamber on the outlet of the toilet system in the position downstream of the water seal arranged between the bowl and the outlet of the toilet system and adjacent to at least a part of a lower half of the outlet pipe of the toilet system.

In further embodiments the method further comprises one or more of the further steps (i) providing a plurality of through openings to comprise a largest size A measured in a direction perpendicular to the longitudinal axis L of the outlet and a largest size B measured in a direction parallel with the longitudinal axis L of the outlet, and where neighboring through openings are arranged spaced apart with a smallest distance C measured in a direction perpendicular to the longitudinal axis L of the outlet and a smallest distance D measured in a direction parallel with the longitudinal axis L of the outlet, and wherein the plurality of openings are arranged in a pattern fulfilling the relations C<A and D<2B, (ii) providing the at least one through opening or the plurality of through openings to comprise a cross sectional shape being polygonal, ellipsoid, circular, with any one or more of a regular, concave, convex or irregular edge, or any combination thereof, (iii) providing the at least one through opening or the plurality of through openings to comprise a largest size A measured in a direction perpendicular to the longitudinal axis L of the outlet and a largest size B measured in a direction parallel with the longitudinal axis L of the outlet, where the size A is between 0.5 mm and 100 mm, and alternatively, or additionally, where the size B is between 0.5 mm and 100 mm, (iv) providing the plurality of through openings to comprise at least five openings, or (v) providing any one or more of (a) a screening device arranged upstream of the at least one through opening or the plurality of through openings, (b) a mass sensor or a weight sensor arranged and configured to monitor a weight of the chamber and to capture at least one signal indicative of a quantity of excretion excreted into the bowl by a user of the toilet system, (c) a flow sensor arranged and configured to monitor a flow of liquid through the chamber and to capture at least one signal indicative of a quantity of excretion excreted into the bowl by a user of the toilet system, and (d) a mounting element adapted for enabling mounting the sensor device on an outlet pipe of a toilet system.

In yet another embodiment, a fluid balance monitoring system and a method for determining and monitoring the fluid balance of a mammalian subject are provided with which becomes possible to determine all inflows and outflows of fluids to and from a subject, measure the inflows and outflows of fluids to and from a subject separately, determine the type and time of occurrence of each inflow and outflow and summarize the fluid balance or change in mass of liquid in a subject over a period of time, such as to provide a precise and comprehensive fluid balance monitoring and an improved or even full understanding of the dynamics of the fluid balance of a subject.

Certain embodiments provide a fluid balance monitoring system and a method for determining and monitoring the fluid balance of a mammalian subject which further simplifies both the practical data registration process and the work process of health professionals when monitoring a mammalian subject. Certain embodiments provide a fluid balance monitoring system which saves time and costs, especially within the professional health care systems, such as hospitals, nursing homes and homecare.

In yet another embodiment, a fluid balance monitoring system for determining and monitoring the fluid balance of a mammalian subject is provided, and the fluid balance monitoring system comprises at least one data processing unit, at least one input unit and at least one display unit. The at least one input unit is configured to receive, from any one or more of a user interface and at least one sensor device, measurement data comprising information relevant for determining and monitoring the fluid balance of a mammalian subject, and transmit the received measurement to the at least one data processing unit. The at least one data processing unit comprises a data processing device, and the at least one data processing unit is configured to receive the measurement data from the at least one input unit, process the received measurement data to achieve output data indicative of the fluid balance of the mammalian subject, and transmit the output data indicative of the fluid balance of the mammalian subject to the at least one display unit. The at least one display unit comprises a data processing device and the at least one display unit is configured to receive the output data indicative of the fluid balance of the mammalian subject, and display the output data indicative of the fluid balance of a subject. The measurement data further comprises information regarding a quantity, a type and a time of occurrence for at least one event relevant for determining and monitoring the fluid balance of the mammalian subject, where the at least one event is any one relevant event leading to an outflow of fluid from the mammalian subject or an inflow of fluid to the mammalian subject, and the at least one data processing unit furthermore is configured to process the received measurement data to achieve output data which is further indicative of a type and a time of occurrence of the at least one event.

Thereby, and especially by providing that the measurement data further comprises information regarding a quantity, a type and a time of occurrence for at least one event relevant for determining and monitoring the fluid balance of a subject, where the at least one event is any one relevant event leading to an outflow of fluid from the subject or an inflow of fluid to the subject, and that the at least one data processing unit furthermore is configured to process the received measurement data to achieve output data which is further indicative of a type and a time of occurrence of the at least one event, a fluid balance monitoring system is provided with which it becomes possible to determine all inflows and outflows of fluids to and from a subject, measure the inflows and outflows of fluids to and from a subject separately, determine the type and time of occurrence of each inflow and outflow and summarize the fluid balance or change in mass of liquid in a subject over a period of time.

Such a system thus enables providing a precise and comprehensive fluid balance monitoring as well as an improved or even full understanding of the dynamics of the fluid balance of a subject. This in turn provides the further advantage of helping professionals to further understand the dynamics of the fluid balance of the subject, to make quicker and more precise diagnoses, and to more closely and precisely monitor subjects with the aim of a faster and better recovery as well as to prevent hospitalizations and other complications that may follow from an unbalanced liquid balance

Furthermore, a fluid balance monitoring system which saves time and costs, especially within professional health care systems, such as at hospitals, is provided herewith. Saving such costs and especially time may also aid in providing the further advantage of helping professionals to make quicker and more precise diagnoses, and to more closely and precisely monitor subjects with the aim of a faster and better recovery.

Also, by providing that the data collection takes place on one unit, the data processing in another unit and the display of data on yet another unit, the fluid balance monitoring system may be centrally managed by the data processing unit. With such a fluid balance monitoring system the data registration process and the display and monitoring process, respectively, may take place on separate units. Thereby, the subject or a caretaker may manage the data collection and registration on one unit, and the subject's fluid balance may be monitored on a different unit. Thus, both the practical data registration process and the work process of health professionals when monitoring a subject is simplified considerably.

In an embodiment, the at least one data processing unit is in data transferring connection with the at least one input unit and the at least one display unit in such a way that all data communication between the at least one input unit and the at least one display unit is lead through the at least one data processing unit. Thereby, it is provided that the fluid balance monitoring system is centrally managed by the data processing unit. This in turn provides for a simplified fluid balance monitoring system with which the data registration process and the display and monitoring process, respectively, may take place on separate units. Thereby, the subject or a caretaker may manage the data collection and registration on one unit, and the subject's fluid balance may be monitored on a different, separate unit. This provides for that the different users of the system are only presented with information relevant for them. Thus, both the practical data registration process and the work process of health professionals when monitoring a subject is simplified even further.

In an embodiment, the measurement data comprises data related to at least one event relevant for determining and monitoring the fluid balance of the mammalian subject, where the at least one event comprises any one or more of: excretion, defecation, urination, intake of food or drink, exercise, perspiration, respiration, sputum secretion, mucosal secretion, output from drain and gastric evacuation. Taking into account all or most relevant such events in the fluid balance monitoring of a subject provides a further improved fluid balance monitoring in terms of precision and comprehensiveness. This in turn further improves the precision in both diagnosing subjects and in monitoring subjects with the aim of a faster and better recovery.

In an embodiment, at least one of the measurement data and the output data is saved in a cloud-based storage or a storage provided in the data processing unit. Thereby, a fluid balance monitoring system is provided with which the output data and measurement data may be kept stored centrally such as to enable access to the data by any authorized display unit at any time. This improves the flexibility of the system.

In a further embodiment, at least one of the measurement data and the output data is associated with or encrypted by an individual identification key. Alternatively, or additionally, at least one of the measurement data and the output data may be saved in a cloud-based storage or a storage provided in the data processing unit together with or encrypted by an individual identification key. Thereby, it is provided that the measurement data and the output data may be associated with a particular subject in a particularly simple manner. Furthermore, it is also ensured that the measurement data and output data is always associated with the correct subject. Thereby, errors which may occur in case of erroneously associating data with a wrong subject may be minimized or altogether avoided.

In an embodiment the individual identification key is an anonymized individual identification key. Thereby, the identification of the subject may be kept anonymous, at least until display on the display unit, such as to conform with relevant regulations, such as the GDPR-regulations in force within the European Union.

Furthermore, any of the three above-mentioned embodiments related to central data storage, and especially the provision of an individual identification key, provides for a system in which unauthorized personnel may be denied access to the data, for instance based on a separate key authorizing access to the data processing unit or based on the identification key or suitable properties associated with such a key, e.g., an associated key for decryption. Such systems are provided with an improved data security.

In an embodiment, the output data comprises at least data indicative of an inflow of liquid to the mammalian subject, an outflow of liquid from the mammalian subject and a change in the fluid balance of the mammalian subject. Thereby, three parameters related to the liquid balance of a subject may be displayed in the display unit. Thereby, a precise and easy to understand monitoring of the fluid balance is obtained, which in turn makes it possible to understand the dynamics of the fluid balance of the subject.

In an embodiment, the display unit is configured to display the output data in a manner showing the data indicative of an inflow of liquid to the mammalian subject, an outflow of liquid from the mammalian subject and a change in the fluid balance of the mammalian subject separately from one another. Thereby, a fluid balance monitoring system is provided with which it becomes possible to not only determine and measure all inflows and outflows of fluids to and from a subject and measure the inflows and outflows of fluids to and from a subject separately, but also to visualise and display the said inflows and outflows separately together with the fluid balance of the subject, such as to provide a precise and comprehensive fluid balance monitoring being easy to interpretate for the viewer, such as a health professional.

In an embodiment, the data processing unit is configured to generate output data and send the output data to the at least one display unit in real time or with predetermined time intervals. Alternatively, or additionally, the display unit is configured to display the output data in real time or with predetermined time intervals. Thereby, real time monitoring of the fluid balance of a subject is enabled in a particularly simple, quick and easy to use manner.

In an alternative embodiment, the invention pertains to a method for determining and monitoring the fluid balance of a mammalian subject, and the method comprises the steps of (a) providing a fluid balance monitoring system according to any one of the above claims and comprising at least one data processing unit with at least one data processing device, at least one input unit and at least one display unit with at least one data processing device, (b) receiving, with the at least one input unit and from any one or more of a user interface and at least one sensor device, measurement data comprising information relevant for determining and monitoring the fluid balance of a mammalian subject, and (c) transmitting, with the at least one input unit, the received the measurement data to the at least one data processing unit, (d) receiving, with the at least one data processing unit, the measurement data from the at least one input unit, (e) processing, with the at least one data processing unit, the received measurement data to achieve output data indicative of the fluid balance of the mammalian subject, (f) transmitting, with the at least one data processing unit, the output data indicative of the fluid balance of the mammalian subject to the at least one display unit, (g) receiving, with the at least one display unit, the output data indicative of the fluid balance of the mammalian subject, and (h) displaying, with the at least one display unit, the output data indicative of the fluid balance of the mammalian subject, the measurement data further comprising information regarding a quantity, a type and a time of occurrence for at least one event relevant for determining and monitoring the fluid balance of the mammalian subject, where the at least one event is any one relevant event leading to an outflow of fluid from the mammalian subject or an inflow of fluid to the mammalian subject, and the method comprising the further step of processing, with the at least one data processing unit, the received measurement data to achieve output data which is further indicative of a type and a time of occurrence of the at least one event.

In some embodiments, the method comprises one or more of the further steps of (i) saving at least one of the measurement data and the output data in a cloud-based storage or a storage provided in the data processing unit, (ii) associating, providing or encrypting at least one of the measurement data and the output data with an individual identification key, (iii) generating, with the data processing unit, output data in real time and sending, with the data processing unit, the output data to the at least one display unit in real time, and (iv) displaying, with the display unit, the output data in real time.

It is noted that the invention relates to all possible combinations of features recited in the claims. Additionally, these and other features of the present invention will be presented in more detail in the following specification of certain embodiments of the invention and the accompanying figures which illustrate by way of example the principles of the invention.

### BRIEF DESCRIPTION OF DRAWINGS

In the following description embodiments of the invention will be described with reference to the schematic drawings, in which

FIG. 1 is a schematic cross-sectional side view of a toilet system according to the invention comprising a first sensor device configured to measure type of excretion and a second sensor device configured to measure quantity of excretion,

FIG. 2 is a schematic illustration of an analysis device for a toilet system according to the invention and shown physically separate from the remaining components of the toilet system shown in FIG. 1,

FIG. 3 is a schematic cross-sectional illustration of a first sensor device according to the invention,

FIG. 4 is a schematic cross-sectional side view of a bowl of a toilet system according to the invention illustrating the difference in sound caused by excretion in dependence of the place in the bowl of the toilet system which the excretion hits.

FIG. 5 is a schematic cross-sectional side view analogous to FIG. 4, where the excretion is urine,

FIG. 6 is an exemplary plot illustrating a signal captured by the first sensor device in a situation according to FIG. 5, the signal being plotted as amplitude A as function of time t,

FIG. 7 is a schematic cross-sectional side view analogous to FIG. 4, where the excretion is faeces,

FIG. 8 is an exemplary plot illustrating a signal captured by the first sensor device in a situation according to FIG. 6, the signal being plotted as amplitude A as function of time t,

FIG. 9 is an exemplary plot illustrating the result of a statistical analysis performed on the signal according to FIG. 6 to provide at any given time the probabilities concerning different types of excretion entering the bowl of the toilet system according to the invention,

FIG. 10 is an exemplary plot illustrating the result of a statistical analysis performed on the signal according to FIG. 8 to provide at any given time the probabilities concerning different types of excretion entering the bowl of a toilet system according to the invention,

FIG. 11 is an exemplary graph illustrating the result of combining the plots of FIGS. 8 to 10 with a measurement captured by the second sensor device of a toilet system according to the invention and performing an analysis on the data to assess the type and the quantity of excretion entering the bowl of a toilet system according to the invention, the result of the analysis being plotted as quantity as function of time, and

FIG. 12 illustrates schematically an embodiment of a method according to the invention.

FIG. 13 illustrates schematically a position of the second sensor device configured to measure quantity of excretion on the seat of a toilet system according to the invention, the remaining parts of the toilet system being omitted for simplicity.

FIG. 14 is a schematic cross-sectional side view of a section of an outlet pipe of a toilet system according to the invention provided with a sensor device comprising a plurality of through openings, of which only one is visible, and a chamber.

FIG. 15 is a schematic cross-sectional side view similar to that of FIG. 14 showing a toilet system according to the invention with an alternative embodiment of the chamber.

FIGS. 16A to 16C schematically illustrate different shapes and patterns of a plurality of through openings of a sensor device of a toilet system according to the invention.

FIGS. 17A and 17B schematically illustrate sizes and distance requirements of a plurality of through openings of a sensor device of a toilet system according to the invention.

FIG. 18 is a schematic cross-sectional view of an outlet pipe of a toilet system according to the invention provided with a sensor device comprising a plurality of through openings, of which only one is visible, a chamber and a mounting element.

FIG. 19 is a schematic cross-sectional side view similar to those of FIGS. 14 and 15 and showing a toilet system according to the invention with an alternative embodiment of the sensor device.

FIG. 20 is a schematic illustration of the liquid inflow and outflow of a subject, particularly a mammalian subject such as a human body.

FIG. 21 is a schematic diagram illustrating an embodiment of a fluid balance monitoring system for determining and monitoring the fluid balance of a mammalian subject according to the invention.

FIG. 22 is a schematic diagram illustrating an embodiment of a method according to the invention.

### DESCRIPTION OF EMBODIMENTS

In the following description, numerous specific details are set forth in order to provide a thorough understanding of the present invention. The present invention may be practiced without some or all of these specific details. In other instances, well known process operations have not been described in detail to not unnecessarily obscure the present invention. While the invention will be described in conjunction with the specific embodiments, it will be understood that it is not intended to limit the invention to the embodiments.

As used herein, the term “excretion”, when used as a noun, is intended to encompass in principle any waste product that is eliminated from a human body, but particularly urine and faeces. Likewise, as used herein the term “excretion” and conjugations thereof, when used as a verb, is intended to encompass the action of expelling in principle any waste product that is to be eliminated from a human body.

As used herein, the term “main axis” is intended to refer to an axis parallel with any one of a base, particularly a base line, and a principal axis, particularly a minor axis, of an object, depending on the specific geometrical shape of the object.

As used herein, the term “liquid pressed through a water seal of the toilet system”, and in particular “pressed through”, is intended to encompass liquid pressed through a water seal of the toilet system irrespective of how, i.e. including not only by an excretion forcing liquid through the water seal, but also overflowing given that it is the force of gravity pressing on the liquid.

As used herein the terms “subject” and “mammalian subject” are intended to encompass in principle any subject for which it is desired to monitor the fluid balance, but particularly such subjects being relevant for the health sector, i.e. mammals such as particularly human beings.

There are several drawbacks to current toilet systems. In certain systems, it is not possible to measure the mass of the bowl as it is constant, or the change in mass only corresponds to the volume required to fill the bowl until it overflows. Also, different events of interest, such as urination and defecation, causes a smaller outflow than e.g. rinsing the bowl by flushing. Likewise, when rinsing the bowl, particulate matter is likely to flow out of the container. Thus, if one wants to measure the events of interest, a selective capturing method may be needed. This may be obtained by means of a toilet system where a sensor device is attached to the outlet of the toilet system downstream of the water seal, whereby in case of overflow all the water is collected through a hole or opening provided in the outlet pipe.

However, several factors affect the design of such a toilet system in order to obtain a reliable and precise measurement result. For instance, providing a too small hole may result in clogging if particulate matter is latched in them, eventually obstructing the flow that were intended for measuring. Another challenge is that very small events of interest, can cause liquid intended for measuring to flow around the hole.

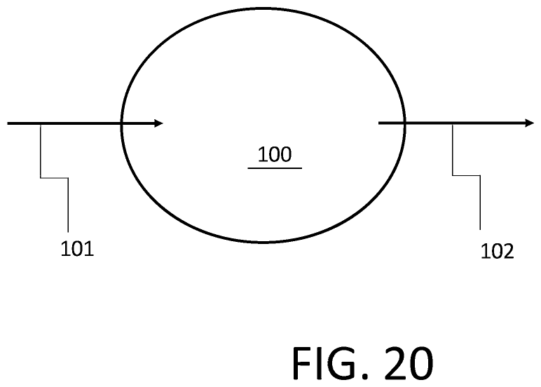

Additionally, the fluid balance of a subject, such as a mammalian or human subject, affects homeostatic processes such as acid-base balance, ion balance, liquid transport and similar within the subject. The fluid balance of a subject 100 is illustrated schematically on FIG. 20. The fluid balance of a subject 100 expresses the change of water in the subject 100, that is the relation between the inflow 101 or volume of liquid flowing into the subject 100 and the outflow 102 or volume of liquid flowing out of the subject 100. The fluid balance of a subject 100 to a given time can be expressed as:

Fluid balance=Inflow−Outflow

The fluid balance of a subject, such as a human subject, is a parameter, which within the health sector is frequently used as one parameter amongst others when diagnosing diseases and monitoring progress of diseases. Liquid can flow into the subject via intake of food or drink or by means of access through bone, vascular system, intestines and even via surfaces if there is a difference in the water vapor pressure, or osmotic pressure, between internally and externally with respect to the subject. Liquid can also flow out of the subject through evaporation (e.g. sweating) from surface areas such as lung tissue or skin tissue, or via expelling excretions such as urine and feces. Further, liquid can flow out of the subject orally via sputum, ulcers, drains, mucosal, and gastric evacuations.

A simple way of measuring the fluid balance of a subject is by monitoring the mass of the subject. Measuring the mass of a subject can be done by weighing the subject. However, the mass will only signify the present status of the subject and thus only provides a snapshot of the fluid balance of the subject. An increase in mass can occur due to increased inflow with a constant outflow, or similarly, a constant inflow and decreased outflow. Likewise, a decrease in mass can occur due to decreased inflow with a constant outflow, or similarly, a constant inflow and an increased outflow.

Furthermore, the type of inflow and outflow is considered, as the water content of different excretions, e.g., faeces and urine, differ. Therefore, a determination of type of outflow may be used to properly assess the amount of water flowing out of a subject. Likewise, for inflow, the water content of a similar mass of different substances consumed, e.g., water and bread, or otherwise flowing into a subject differ. Therefore, a determination of the type of inflow is also a factor.

Thus, it is desired to measure the inflow and outflow separately and to enable determining the type and time of occurrence of each inflow and outflow to understand the dynamics of the fluid balance of the subject. Also, it is desired to summarize the fluid balance or change in mass of liquid in a subject over a period of time, t, where the change in mass of liquid equals the sum of liquid inflows subtracted the sum of liquid outflows in that period of time and can be expressed as

Σ(Fluid balance,t)=Σ(Inflow,t)−Σ(Outflow,t),

where the notation Σ(x, t) denotes the sum of the parameter x over the time t. The fluid balance can be expressed as an absolute value or as a percentage of subject mass, e.g. a negative percentage if outflow is larger than inflow, 0% if inflow and outflow are equal or a positive percentage if inflow is larger than outflow.

To fully understand the dynamics of the fluid balance of a subject, all inflow and outflow may be determined. Ideally, this determination would be done autonomously. However, the technological environments do not always allow this. In these environments, it may be necessary to perform registrations of time and type for each time an inflow or an outflow occurs. Typically, this is done using paper and pen, which is however time consuming and unhygienic and very prone to registration errors.

It is thus still further desired to provide a fluid balance monitoring system and a method for determining and monitoring the fluid balance of a subject alleviating at least some of the above-mentioned and other drawbacks.

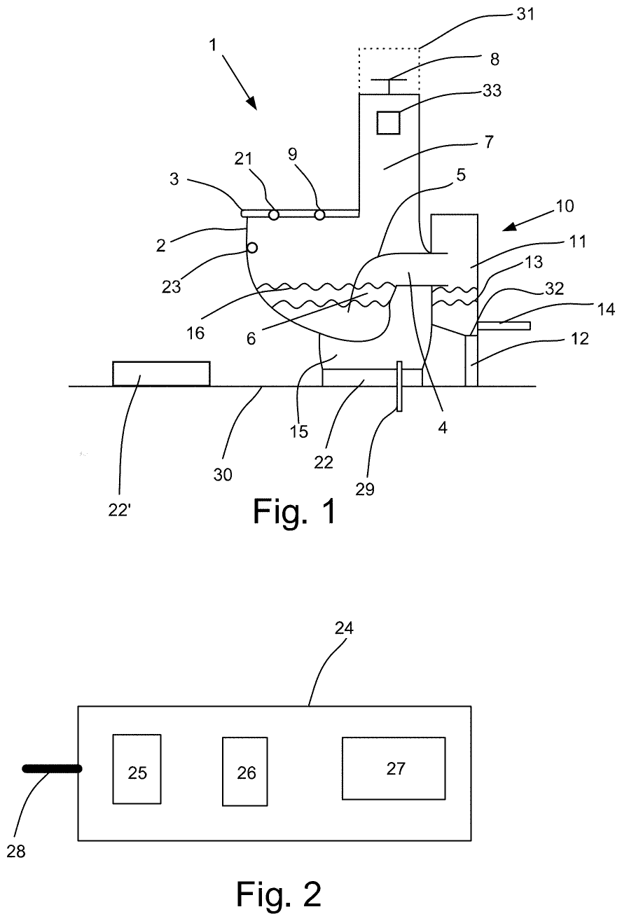

Referring initially to FIGS. 1 and 2, a first embodiment of a toilet system 1 according to the invention is shown. The toilet system 1 comprises a bowl 2, an outlet 4 and a seat 3. The outlet 4 further comprises an outlet pipe 5 with a water seal, water trap or U-bend 6. The toilet system 1 may further comprise a toilet tank or flushing cistern 7 with a device 8 adapted for causing the toilet to flush when operated. The device 8 may be an actuator such as a press button or a pull button or a lever. The toilet system 1 may further comprise a basis 15 for connection of the toilet system 1 to a floor 30.

The toilet system 1 comprises a first sensor device 9 and a second sensor device 10. The toilet system 1 may also optionally comprise further sensor devices 11.

The toilet system 1 may be a new separate toilet system 1. Alternatively, the first sensor device 9 and the second sensor device 10 may be mounted on an existing toilet such as to provide a retrofitted toilet system 1. A toilet system 1 according to the invention may thus be provided by retrofitting an existing toilet by providing a first sensor device 9 and a second sensor device 10 and mounting the first sensor device 9 and the second sensor device 10 on the existing toilet.

Generally, the first sensor device 9 may be configured to capture a signal indicative of a type of excretion excreted into the bowl 2 by a user of the toilet system 1. The first sensor device may be further configured to transmit the captured signal to an analysis device 24, which is described further below with reference to FIG. 2.

The first sensor device 9 may be adapted for measuring sound. The first sensor device 9 may be a vibration sensor, such as an acoustic sensor or a microphone. Suitable types of microphones include dynamic microphones, piezoelectric microphones, crystal microphones, fiber-optic microphones and MEMS microphones. The first sensor device 9 may be wired or wireless.

The first sensor device 9 is arranged in or on the seat 3. Alternatively, the first sensor device 9 may be arranged in the bowl 2 or on an inner surface of the bowl 2. In any event the first sensor device 9 may be arranged in a position enabling the first sensor device 9 to capture the sounds caused by or in connection with a user excreting an excretion, e.g. voiding his or her bowel and/or bladder, into the bowl 2. The first sensor device 9 may be arranged above the upper water level 16 in the bowl, but may alternatively also be arranged below the upper water level 16 in the bowl.

Using a first sensor device 9 adapted for measuring sound and thus capturing a sound signal indicative of a type of excretion excreted into the bowl 2 by a user of the toilet system 1 provides for a high degree of detail in the data captured. Different types of excretion, particularly urine and faeces, respectively, thus generate different sound profiles of the sound signal. It may even be possible through careful analysis to discern between different consistencies of faecal excretions, e.g. solid faeces and diarrhoea, which may be of importance for diagnostic purposes. The sound profile further changes in accordance with the part of the bowl hit by the excretion. As is illustrated in FIG. 4, a sound signal with one sound profile results from the excretion 17 hitting the bowl 2 directly (arrow 18), while a sound signal with another sound profile results from the excretion 17 hitting the water surface 16 in the bowl 2. Furthermore, using a first sensor device 9 adapted for measuring sound and thus capturing a sound signal even makes it possible to identify and remove, during analysis, unwanted data, such false positives resulting from for instance paper or vomit or something else different from excretion of faeces or urine being excreted into the bowl 2 or even the user merely passing gas, or nothing happening at all.

The at least one first sensor device 9 may also comprise or be any one or more of a pressure sensor, a radar, an image capturing device, a distance sensor, a LIDAR, an acoustic distance sensor, and a flow rate sensor, such sensors also being capable of providing a signal indicative of a type of excretion excreted into the bowl 2 by a user of the toilet system 1.

Generally, the second sensor device 10 may be configured to measure a volume of liquid indicative of a quantity of excretion excreted into the bowl 2 by a user of the toilet system 1. The second sensor device 10 may further be configured to transmit the captured signal to an analysis device 24 (FIG. 2). The second sensor device 10 may be arranged and configured to measure the amount of liquid forced through the water seal or U-bend 6 when a user voids his or her bowel and/or bladder into the bowl 2. In virtue of Archimedes' law, the amount of liquid forced through the water seal or U-bend 6 when a user voids his or her bowel and/or bladder into the bowl 2 is equivalent, or at least close to equivalent depending on the buoyancy of the excreted material, to the quantity of excretion excreted into the bowl 2 of the user.

The second sensor device 10 is in the embodiment shown in FIG. 1 arranged in the outlet 4 of the toilet in a position downstream of the water seal 6. The second sensor device 10 comprises a chamber 11 for collecting the water 13 forced through the water seal 6 as a result of a user depositing an excretion in the bowl 2 of the toilet system 1. The second sensor device 10 further comprises a measuring unit 14 configured to measure the quantity of liquid 13 collected in the chamber 11. The second sensor device 10 further comprises an outlet 12 arranged in a bottom area of the chamber 11 and connected to a sewer system or the like. The second sensor device 10 may still further comprise a closure mechanism 32, such as a valve, a flap or a shutter, configured to be closed while the measuring unit 14 measures the quantity of liquid 13 collected in the chamber 11 and to be opened subsequently to allow the chamber 11 to be emptied and the toilet system 1 to be flushed when the toilet system 1 is flushed.

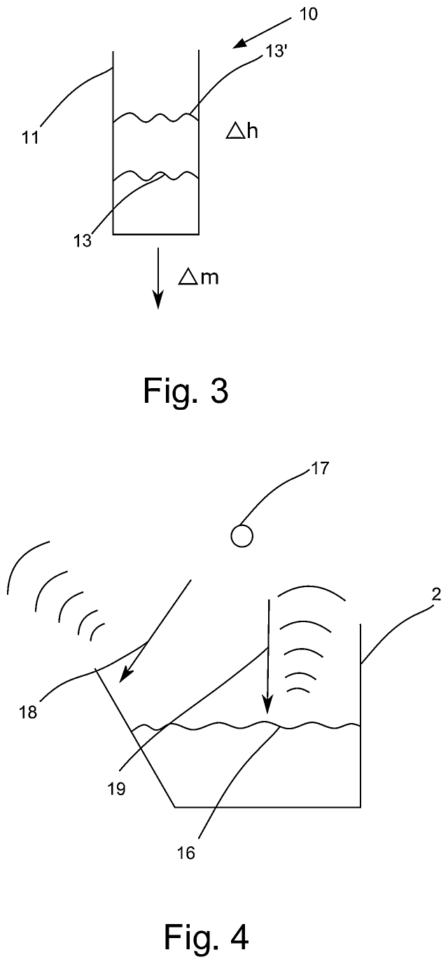

Referring also to FIG. 3, the quantity of liquid collected in the chamber 11 may for instance be measured as a difference, ΔH, in depth or water level between a depth 13 before and a depth 13′ after an excretion has been excreted into the bowl 2 of the toilet system 1. The quantity of liquid collected in the chamber 11 may also be measured as a difference, ΔM, in mass of the water in the chamber with contained water before and after an excretion has been excreted into the bowl 2 of the toilet system 1, respectively, or as a difference in volume, ΔV, of water exiting the chamber 11 when the closure mechanism 32 is opened after an excretion has been excreted into the bowl 2 of the toilet system 1.

The second sensor device 10 may alternatively be arranged in the bowl 2 or the outlet 4. Such an embodiment is especially advantageous in case of retrofitting an existing toilet with a first sensor device 9 and a second sensor device 10 since it makes the installation of the second sensor device 10 particularly easy and cost efficient. In any event the second sensor device 10 is arranged in a position enabling the second sensor device 10 to measure the amount of liquid forced through the water seal or U-bend 6 when a user excretes an excretion into the bowl 2.

The second sensor device 10 may be or comprise any one or more of a weight sensor, a level sensor and a flow sensor. By level sensor one may understand any type of distance measuring device being mounted in a fixed position, here with respect to the surface of the liquid collected in the chamber 11, whether above or below said surface.

The measuring unit 14 of the second sensor device 10 may comprise a flow sensor, a weight sensor, a depth gauge, a volume sensor or the like.

The toilet system 1 may in some embodiments also comprise one or more further sensor devices, such as sensor device 23 and/or sensor devices 22 and 22′ shown on FIG. 1. The one or more further sensor devices 23 may comprise any one or more of a pressure sensor, a radar, an image capturing device, and a flow rate sensor, such sensors being capable of providing further data or capturing further signals indicative of a type of excretion excreted into the bowl 2 by a user of the toilet system 1.

The one or more further sensor devices 22, 22′ may be weight sensors, each configured to capture a signal indicative of the weight of the user before and after depositing an excretion into the bowl 2 of the toilet system 1. The further sensor device 22 represents a weight sensor arranged under the toilet system 1, particularly under the bowl 2 and/or the basis 15 of the toilet system 1. The further sensor device 22′ represents a weight sensor arranged under the feet of a user sitting on the seat 3 of the toilet system 1. The further sensor devices 22 and 22′ may both be provided and supplement one another. Alternatively, only one of the further sensor devices 22 and 22′ may be provided. It is also feasible to arrange such a weight sensor in or under the seat 3.

The toilet system 1 may further comprise an actuator 33 configured to allow a user to indicate a type of excretion excreted or to be excreted into the bowl 2 by the user of the toilet system 1. The actuator 33 may further be configured to transmit a signal indicative of the user's indication to the data analysis device 24 (FIG. 2). The actuator 33 may for instance be a button, a lever or a touch screen.

The toilet system 1 may further comprise an actuator 21 configured to allow a user to activate the toilet system 1 and/or the data analysis device 24 (FIG. 2) before use of the toilet system 1. The actuator 21 may for instance be a pressure switch or a touch sensor arranged in the seat 3 such as to be automatically actuated when a user sits on the seat 3. Alternatively, the actuator 21 may be a button, a lever, a touch screen, a motion sensor or another suitable type of sensor. In the latter case it is also feasible that the actuators 21 and 33 may be integrated with or arranged next to one another, for instance by means of the same touch screen. Another alternative is an actuator 21 adapted for electronically recognizing, e.g. by means of face recognition, a person or patient for whom measurements of excretion output is desired.

The toilet system 1 may further comprise a connection 29 to a data analysis device 24. Referring to FIG. 2, the data analysis device 24 likewise comprises a connection 28 to the toilet system 1. The connections 28 and 29 may be wired or wireless. Also, the data analysis device 24 may be an external device or it may be integrated with the toilet system, for instance but not limited to on top of the cistern 7 as indicated by the box 31 shown in FIG. 1 with dotted line.

The data analysis device 24 may comprise a data processing unit 25, a data storage unit 26 and a data visualization unit 27. The data visualization unit 27 may for instance be a display. The data visualization unit 27 is an optional unit.

The data analysis device 24 may be integrated in or with another component of the toilet system 1, such as the cistern 7 or the bowl 2, or where provided the second sensor device 10. Alternatively, the data analysis device 24 may be physically separated from the remaining parts of the toilet system, an example being a suitable type of computer, e.g. placed on a table or mounted on a wall, connected to the first sensor device 9, and where provided the second sensor device 10 and/or the further sensor devices 22, 22′, 23, to enable data transfer to and from the said sensor devices.

The data analysis device 24 may be configured to receive one or more signals captured using the first sensor device 9 and, where provided, the second sensor device 10 and/or the further sensor devices 22, 22′, 23 and transmitted by the first sensor device 9 and, where provided, the second sensor device 10 and/or the further sensor devices 22, 22′, 23. The data analysis device 24 may be further configured to analyse, using the data processing unit 25, the received signals such as to produce a data output indicative of the type of excretion and/or the quantity of excretion excreted into the bowl 2 by a user of the toilet system 1.

The data analysis device 24 may be further configured to display, using the data processing unit 25 and on the data visualization unit 27, a data output indicative of the type and/or the quantity of excretion excreted into the bowl 2 by a user of the toilet system 1. The data analysis device 24 may further be configured to store, in the data storage device 26, the received signals and/or the produced data output indicative of the type of excretion and/or the quantity of excretion excreted into the bowl 2 by a user of the toilet system 1. The data storage device 26 may further contain data usable for performing a comparison with data retrieved from the received signals in order to further improve the certainty of the determination of the type of excretion excreted into the bowl 2 by a user of the toilet system 1. Such data may e.g. be data from previous analyses and/or data retrieved from other sources, such as other similar toilet systems. The data may be stored in a database provided in the data storage device 26.

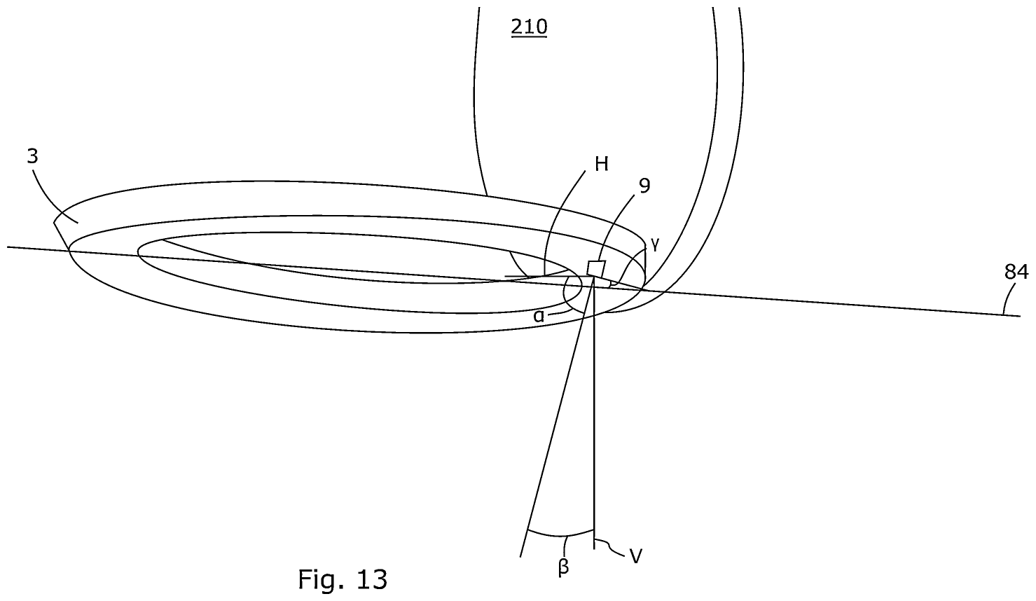

Referring again to FIGS. 1 and 2, the toilet system 1 in a particular embodiment comprises a first sensor device 9 and a second sensor device 10. The first sensor device 9 is an image capturing device. The first sensor device 9 is arranged in or on the seat 3 of the toilet system 1. In any event the first sensor device 9 is arranged in a position enabling the first sensor device 9 to capture images showing excretion, e.g. by a user voiding his or her bowel and/or bladder, and/or other things, e.g. toilet paper, being disposed into the bowl 2. The first sensor device 9 may be arranged above the upper water level 16 in the bowl. Referring also to FIG. 13, the first sensor device 9 is arranged such as to point downwards at an angle α of between 70 and 80 degrees, such as an angle α of 75 degrees, with the horizontal H, or in other words an angle β of between 20 and 10 degrees, such as an angle β of 15 degrees, with the vertical V. Thus, an angle of 90 degrees in this context corresponds to the vertical V or the direction of gravity. Furthermore, the first sensor device 9 is located outside the central axis A of the seat 3 and is rotated around the direction of gravity or vertical V to point in an angle γ of between 25 and 25 degrees, such as an angle γ of 30 degrees, with the central axis A of the seat 3.

The second sensor device 10 may be arranged in the outlet 4 of the toilet system 1 in a position downstream of the water seal 6. The second sensor device 10 comprises a chamber 11 for collecting the water 13 forced through the water seal 6 as a result of a user depositing an excretion in the bowl 2 of the toilet system 1. The second sensor device 10 further comprises an outlet 12 arranged in a bottom area of the chamber 11 and connected to a sewer system or the like. Alternatively, the second sensor device 10 may comprise a so-called outlet pump, i.e. an outlet (not shown on FIG. 1) arranged in a top area of the chamber 11 and connected to a sewer system or the like as well as a pumping device arranged and adapted for emptying the chamber 11 by pumping the contents of the chamber 11 into the outlet and thus into the sewer system. The second sensor device 10 furthermore comprises a measuring unit 14 in the form of a weight sensor arranged and adapted to measure the weight of the chamber 11. In this way, the second sensor device 10 takes advantage of the fact that due to the construction of the water seal 6 and due to Archimede's law, the amount of liquid forced through the water seal 6 of the toilet system 1 is equivalent to the amount of excretion disposed into the bowl 2 of the toilet system 1.

The second sensor device 10 may still further comprise a closure mechanism 32, such as a valve, a flap or a shutter, configured to be closed while the measuring unit 14 measures the weight of the chamber 11 and the liquid 13 collected in the chamber 11 and to be opened subsequently to allow the chamber 11 to be emptied and the toilet system 1 to be flushed when the toilet system 1 is flushed.

The data analysis device 24 (cf. FIG. 2) is in this particular embodiment configured to receive one or more signals captured using the first sensor device 9 and the second sensor device 10 and transmitted by the first sensor device 9 and the second sensor device 10. The data analysis device 24 is further configured to analyse, using the data processing unit 25, the received signals such as to produce a data output indicative of the type of excretion and/or the quantity of excretion excreted into the bowl 2 by a user of the toilet system 1. To this end, the data analysis unit 25 and the data processing unit 25 uses suitable image analysis and/or image recognition software.

The analysis to determine a type of excretion or deposition into the bowl 2 of the toilet system 1 may be completed using a statistical method, such as a neural network, provided in or to the data analysis device 24. The first step for doing the training, or calibration, of the neural network, is to acquire relevant training data. To this end, a sampling of several thousand toilet visits using the above-described sensors has been conducted. The second step is to extract exemplary data for type determination. In this case, the exemplary data is data from the first sensor device 9 which may depict one or more of, e.g., an empty bowl, urine, feces, and paper. The extraction is performed manually by a trained person. The training ensures is that there is provided clear definitions of each category, e.g., empty bowl 2 containing only water, or bowl 2 with urine, feces, and/or paper. The definitions, their interpretation, and the final type recognition are closely linked. The extraction process comprises naming computer directories, naming the relevant categories, and subsequently moving relevant imaging data to the corresponding directory. The statistical model can then be calibrated to classify data from the first sensor device 9 to belong to one of the different categories, for instance by means of the previously described computer directories. Once the statistical model is calibrated, it can organize imaging data as it arrives from the first sensor device 9. Therefore, a time stamped type determination can also be obtained.

The first sensor device 9 and the data analysis device 24 can now be used to tell if there is urine or feces in the bowl 2. The total quantity excreted, as measured by the second sensor device 10, can then be mapped to a category, particularly either urine, feces, or a combination. Changes in total quantity can also be mapped to each category, thus giving the quantity of urine and feces separately. However, in cases where a more considerable amount of feces arrives before urine, it may be difficult to recognize the urine precisely. To correct this error, the same processes can be repeated for data indicative of the active excretory action, e.g., urination and defecation. In principle, both imaging and audio can be input for the excretory action determination.

### Experiment and Example

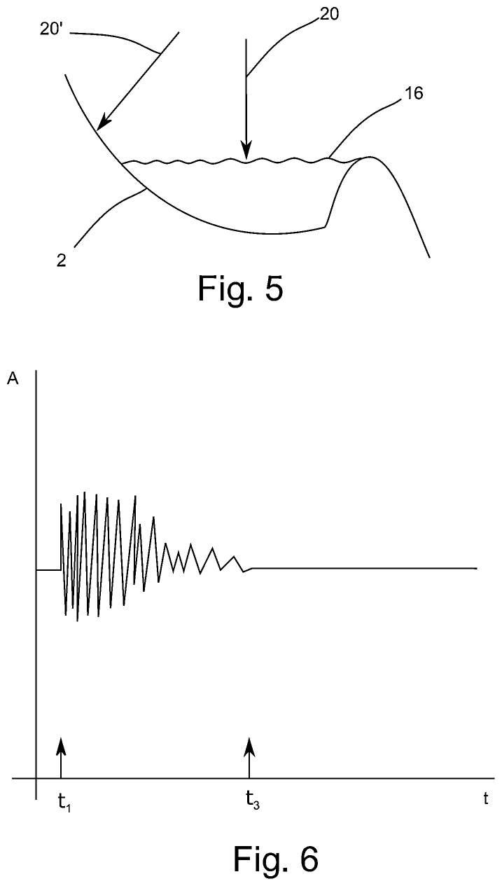

Turning now to FIGS. 5 to 11 a general example of data capture and analysis using a toilet system 1 according to the present invention and as described above will be given. In order to enable identification of the type of excretion as described below, sound capture measurements of more than one thousand different excretions were made to provide background data for comparison purposes and statistical analysis. The background data may be stored in a database, which may be provided in the storage device 26 of the analysis device 24 (FIG. 2). Further data may be added to the database as new measurements are made. The experiment has shown that it is in fact possible to determine the type of excretion based on sound measurements of the excretion with a sufficiently high certainty to be usable in practice. An analogous or similar approach may be made to provide background quantity information for comparison purposes and statistical analysis.

FIG. 5 illustrates a bowl 2 of a toilet system 1 according to the invention into which a user excretes urine 20, 20′, and thus a thin, liquid excretion, directly onto the surface of the bowl 2 and into the water 16 in the bowl, respectively.

FIG. 6 illustrates a transform of the captured sound signal, or more precisely a plot of the signal amplitude A as a function of elapsed time t of a sound signal captured by means of the first sensor device 9 of the toilet system 1 and corresponding to the excretion illustrated in FIG. 5. As may be seen the sound profile extends over a relatively long period of time Δt=t3−t1. At t=t1, the amplitude of the sound signal is large, possibly peaking, and as time elapses towards t=t3, the amplitude gradually decreases towards zero. Such an acoustic image is characteristic for the excretion of urine.

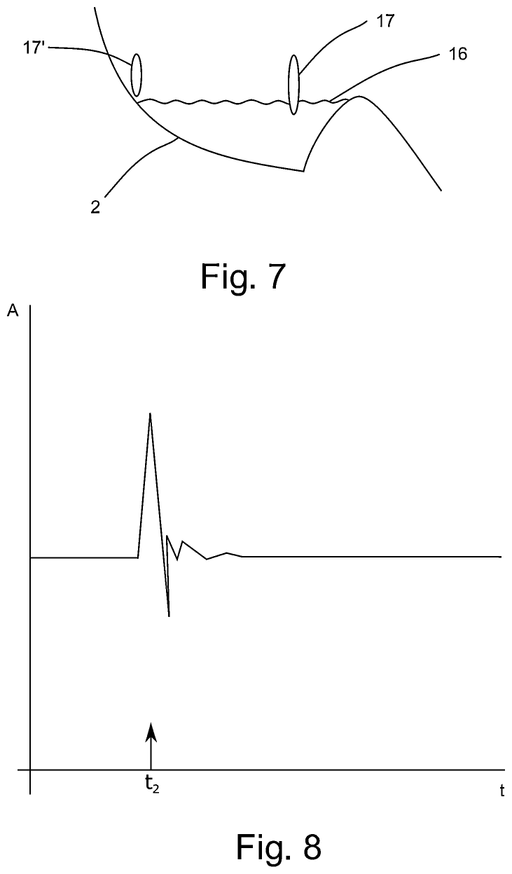

FIG. 7 illustrates a bowl 2 of a toilet system 1 according to the invention into which a user excretes faeces 17, 17′, and thus a relatively solid excretion, directly onto the surface of the bowl 2 and into the water 16 in the bowl, respectively.

FIG. 8 illustrates a transform of the captured sound signal, or more precisely a plot of the signal amplitude A as a function of elapsed time t of a sound signal captured by means of the first sensor device 9 of the toilet system 1 and corresponding to the excretion illustrated in FIG. 7. As may be seen the sound profile extends over a short period of time around t=t2, being the time of impact of the faeces with the water 16 of the surface of the bowl 2, as the case may be. As may be seen, the amplitude of the sound signal increases and decreases rapidly on each side of and close to t=t2. Such an acoustic image is characteristic for the excretion of faeces.

Thus, objects with different characteristics and/or hitting surfaces with different characteristics, and/or being dropped into a container—such as the bowl 2—in different ways, will produce different vibrations and therefore different sounds and acoustic images.

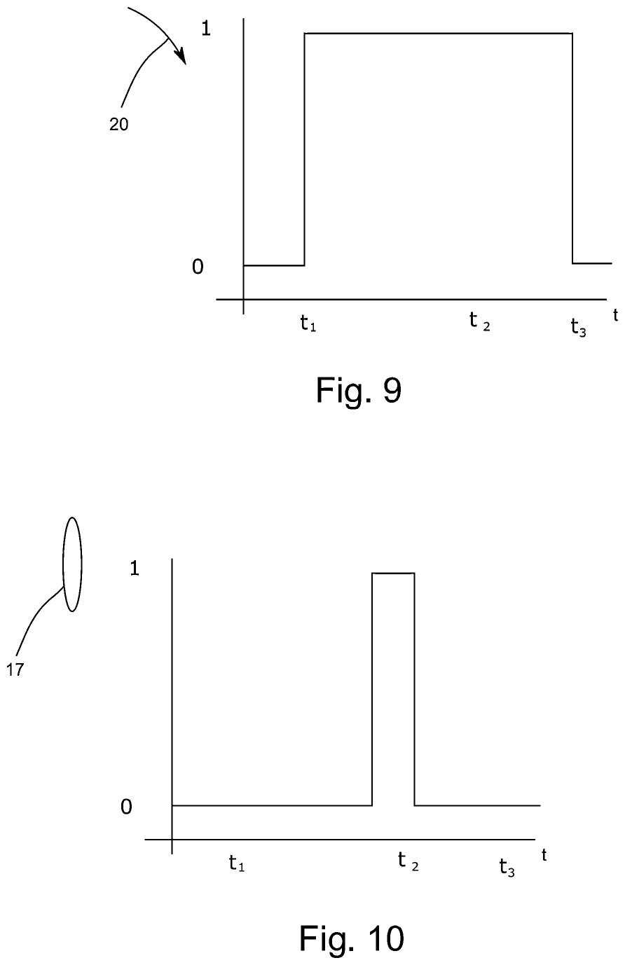

The transforms or plots shown in FIGS. 6 and 8, respectively, may now be analysed, such as analysed statistically, for instance in a neural network, a principal component analysis (PCA), a decision tree or a clustering. Thereby, the probabilities concerning different objects, solids or liquids excreted into the bowl 2 of the toilet system 1 at any given time may be provided. The result of such an analysis of the transforms or plots shown in FIGS. 6 and 8, respectively, are shown in FIGS. 9 and 10, respectively.

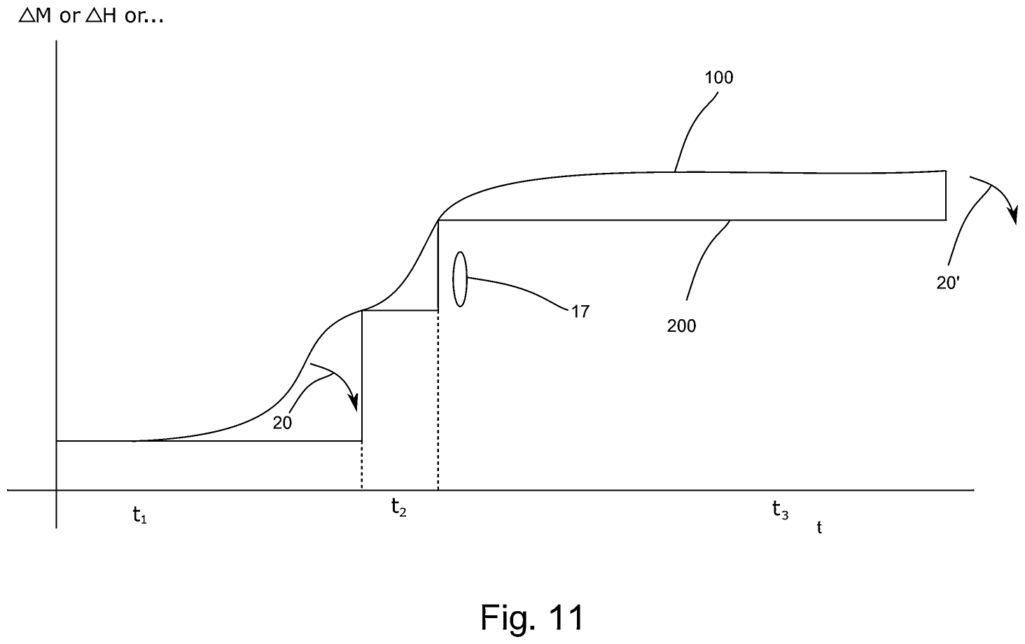

Finally, as illustrated in FIG. 11, the probabilities shown in FIGS. 9 and 10 may, combined with the measures of mass or liquid level of the container or any connected container obtained using the second sensor device 10, be used to assess how much of an object, solid or liquid has entered the bowl 2 of the toilet system 1 as well as when it entered the bowl 2 of the toilet system 1. FIG. 11 illustrates an example of a visualization of the result of such an analysis. Curve 100 is a plot of the change in water level (ΔH), in mass (ΔM), in volume (ΔV) or in flow rate according to measurement principle used as a function of time elapsed as indicated by the signal captured by the second sensor device 10. Curve 200 indicates the accumulated mass or quantity of the excretions. The inserted symbols 20, 17 and 20′ indicates the result of the statistical analysis made on the data provided by the first sensor device 9 and providing probabilities as described above in relation to FIGS. 6 and 8, and thus the identified respective types of excretion.

As shown in FIG. 11, the exemplary analysis reveals that with a high probability in the time interval between t=t1 and t=t2, a first amount of urine 20 was excreted into the bowl 2, in the time interval just around t=t2, an amount of faeces 17 was excreted into the bowl 2, and in the time interval between t=t2 and t=t3, a second amount of urine 20′, being smaller than the first amount of urine 20, was excreted into the bowl 2.

The combination of a sound signal provided by the first sensor device 9 and a further signal provided by the second sensor device 10 will increase the True Negative Rate of the analysis. For instance, sounds such as those of flatulence is not always associated with a change in mass or volume of the container and may thus be removed upon identification.

Compared to only using the mass or volume, the experiments have shown that a better certainty can be obtained as to what liquid or solid is entering the container. For instance, it is possible to differ between liquids poured in at the same rate, whether the liquids hit different parts, but also if the width of the liquid stream is different but the flow the same. The same applies to solids.

Furthermore, the experiments have shown that different types of excretion, particularly urine and faeces, respectively, be identified. It is even possible to discern between different consistencies of faecal excretions, e.g. solid faeces and diarrhoea, which may be of useful for diagnostic purposes. It has further been shown to be possible to identify and remove, during analysis, false positives resulting from for instance paper or vomit or something else different from excretion of faeces or urine being excreted into the bowl 2 or even the user merely passing gas, or nothing happening at all.

Referring again to FIG. 1, the toilet system 1 comprises a sensor device 10 configured to capture a signal indicative of a quantity of excretion excreted into the bowl 2 by a user of the toilet system 1 and an optional sensor device 9 configured to capture a signal indicative of a type of excretion excreted into the bowl 2 by a user of the toilet system 1. The toilet system 1 may also optionally comprise further sensor devices 11.

The toilet system 1 may be a new separate toilet system 1. Alternatively, the sensor devices 9 and 10 may be mounted on an existing toilet system such as to provide a retrofitted toilet system 1. A toilet system 1 according to the invention may thus be provided by retrofitting an existing toilet system by providing a sensor device 9 and a sensor device 10 and mounting the sensor devices 9 and 10 on the existing toilet system. The at least one through opening or the plurality of through openings to be described further below may be provided in the existing outlet pipe. It is noted that in the context of the present invention, the sensor device 9 is optional. It is furthermore noted that the position and type of toilet system 1 may influence on the retrofitting possibilities. For instance, toilet systems with a P-type trap and toilet systems of the wall-hung type may easily be retrofitted.

Generally, the optional sensor device 9 is configured to capture a signal indicative of a type of excretion excreted into the bowl 2 by a user of the toilet system 1. The sensor device is further configured to transmit the captured signal to an analysis device.

The sensor device 9 may be adapted for measuring sound. The sensor device 9 may be a vibration sensor, such as an acoustic sensor or a microphone. Suitable types of microphones include dynamic microphones, piezoelectric microphones, crystal microphones, fiber-optic microphones and MEMS microphones. The sensor device 9 may be wired or wireless.

The sensor device 9 is arranged in or on the seat 3. Alternatively, the sensor device 9 may be arranged in the bowl 2 or on an inner surface of the bowl 2. In any event the sensor device 9 is arranged in a position enabling the sensor device 9 to capture the sounds caused by or in connection with a user excreting an excretion, e.g. voiding his or her bowel and/or bladder, into the bowl 2. The sensor device 9 may be arranged above the upper water level 16 in the bowl but may alternatively also be arranged below the upper water level 16 in the bowl.

The at least one sensor device 9 may also comprise or be any one or more of a pressure sensor, a radar, an image capturing device, a distance sensor, a LIDAR, an acoustic distance sensor, and a flow rate sensor, such sensors also being capable of providing a signal indicative of a type of excretion excreted into the bowl 2 by a user of the toilet system 1.

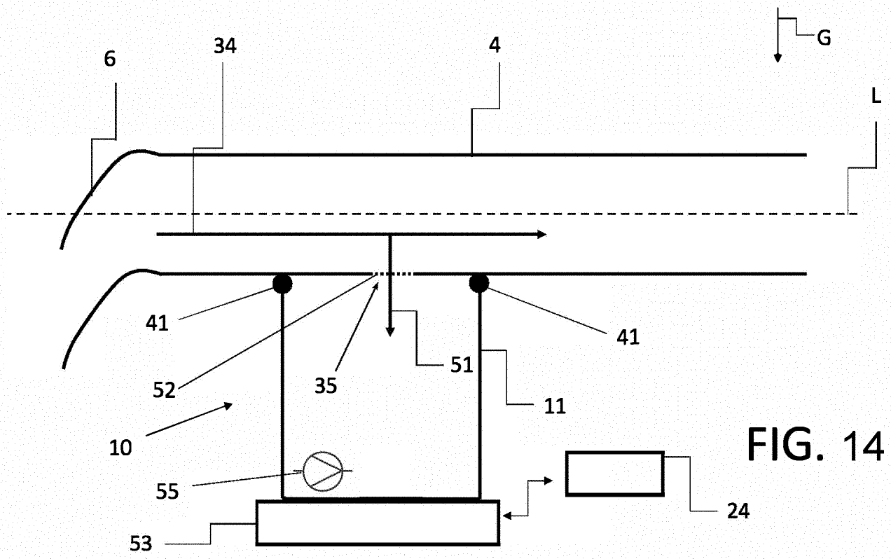

Generally, the sensor device 10 is configured to measure a volume of liquid indicative of a quantity of excretion excreted into the bowl 2 by a user of the toilet system 1. The sensor device 10 may further be configured to transmit the captured signal to an analysis device 24 (FIG. 14). The sensor device 10 may be arranged and configured to measure the amount of liquid forced through the water seal or U-bend 6 when a user voids his or her bowel and/or bladder into the bowl 2. In virtue of Archimedes' law, the amount of liquid forced through the water seal or U-bend 6 when a user voids his or her bowel and/or bladder into the bowl 2 is equivalent, or at least close to equivalent depending on the buoyancy of the excreted material, to the quantity of excretion excreted into the bowl 2 of the user.

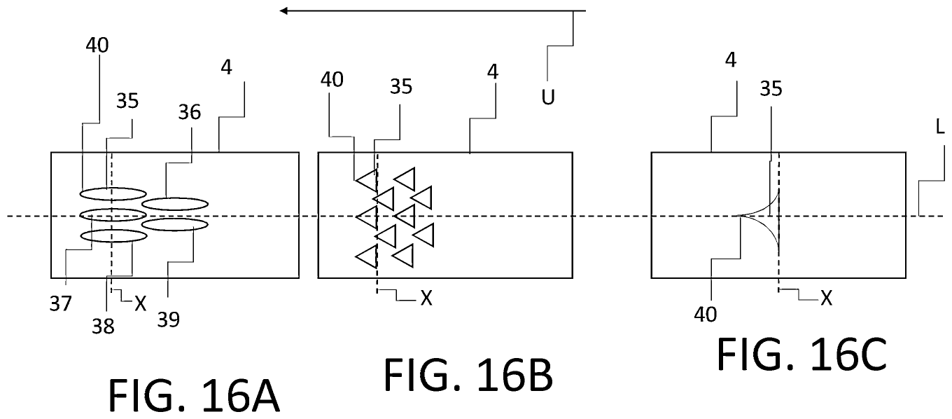

Generally, and referring also to FIGS. 14, 15 and 16A-16B the sensor device 10 comprises at least a chamber 11 and a plurality of through openings 35, 36, 37, 38, 39. Alternatively, and referring to FIG. 16C, the sensor device may also comprise at least a chamber 11 and one through opening 35. Generally, the sensor device 10 is arranged in the outlet 4 of the toilet system 1 in a position downstream of the water seal 6.

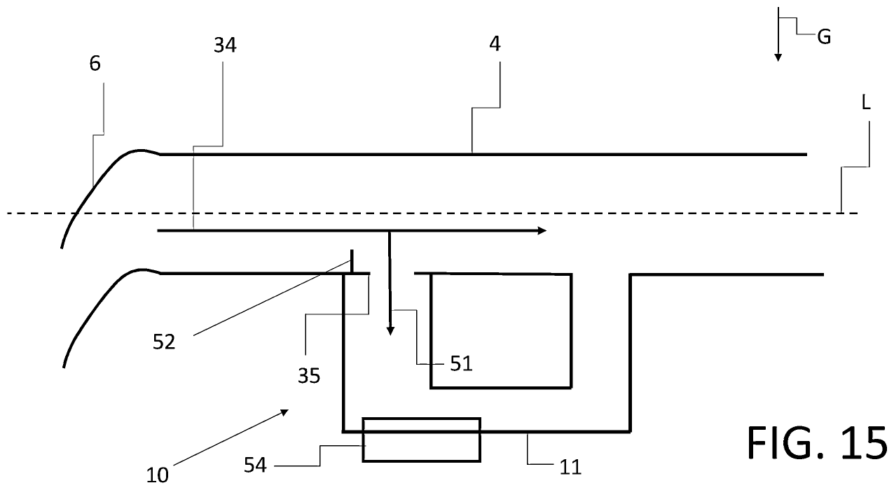

The at least one through opening 35 or the plurality of through openings 35, 36, 37, 38, 39 are provided in the outlet 4 of the toilet system 1 in a position downstream of a water seal 6 arranged between the bowl 2 and the outlet 4 of the toilet system 1, particularly at a lower half of the outlet 4 of the toilet system 1, such as to allow liquid being forced through the water seal 6 to flow into the chamber 11. This may be seen from FIGS. 14 and 15, in which the direction of flow 34 in the outlet pipe 4 is shown, and revealing that a part of the water seal 6 may be seen at the left hand side of FIG. 14. The direction of flow 51 of the liquid diverted through the openings 35, 36, 37, 38, 39 into the chamber 11 is also shown on FIGS. 14 and 15.

The chamber 11 is provided for collecting the water 13 (FIG. 1) or liquid forced through the water seal 6 as a result of a user depositing an excretion in the bowl 2 of the toilet system 1. The chamber 11 is arranged on the outlet 4 of the toilet system 1 in essentially the same position downstream of the water seal 6 as the plurality of through openings 35, 36, 37, 38, 39. The chamber 11 is, just as the plurality of through openings 35, 36, 37, 38, 39, further arranged adjacent to a lower half of the outlet pipe 4 of the toilet system 1. Thereby, the water 13 forced through the water seal 6 as a result of a user depositing an excretion in the bowl 2 of the toilet system 1 may enter the chamber 11 by the action of gravity. The direction of gravity is indicated on FIGS. 14 and 15 by the arrow G.

As shown on FIG. 14, the chamber 11 may be a receptacle. The sensor device 10 may in such an embodiment further comprise a mass sensor or a weight sensor 53 arranged and configured to monitor a weight of the chamber and to capture at least one signal indicative of a quantity of excretion excreted into the bowl by a user of the toilet system. Alternatively, or additionally the sensor device 10 may further comprise a flow sensor, such as e.g. an acoustic flow sensor arranged and configured to monitor a weight of the chamber and to capture at least one signal indicative of a quantity of excretion excreted into the bowl by a user of the toilet system.

Alternatively, and as shown on FIG. 15, the chamber 11 may be a pipe shaped chamber 11 having one end connected to the outlet pipe 4 at the plurality of through openings 35, 36, 37, 38, 39 and another end connected to the outlet pipe 4 at a suitable position further downstream in order to lead the contents of the chamber 11 back into the outlet pipe 4. The sensor device 10 may in such an embodiment further comprise a flow sensor 54 arranged and configured to monitor a weight of the chamber and to capture at least one signal indicative of a quantity of excretion excreted into the bowl by a user of the toilet system. The flow sensor 54 may e.g. be an acoustic flow sensor providing the advantage of enabling monitoring the flow through the chamber 11 without the flow sensor having to be placed in the chamber 11 potentially obstructing the flow to some extent. Alternatively, or additionally the sensor device 10 may further comprise a mass sensor or a weight sensor arranged and configured to monitor a weight of the chamber and to capture at least one signal indicative of a quantity of excretion excreted into the bowl by a user of the toilet system.

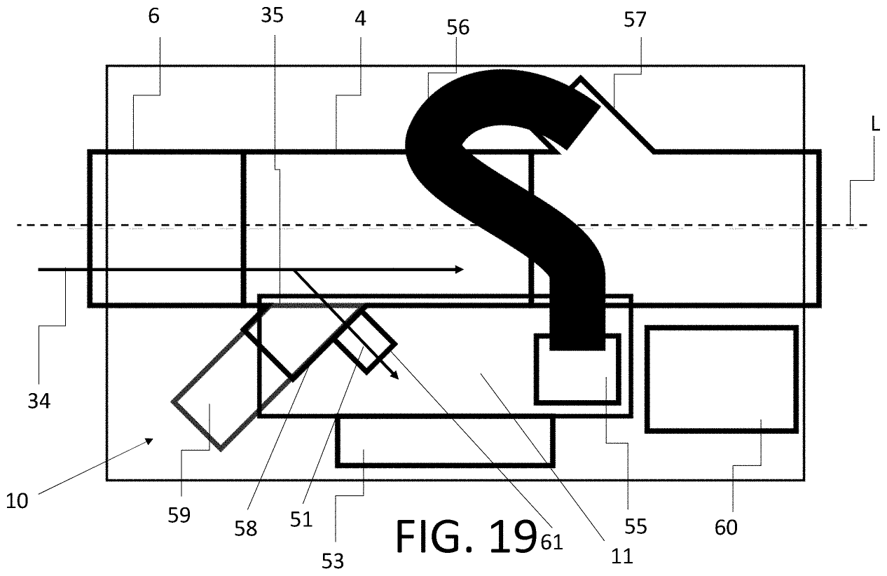

The chamber 11 may furthermore optionally be provided with a pump 55 for evacuating the contents of the chamber 11 back into the outlet pipe 4—cf. FIG. 19.

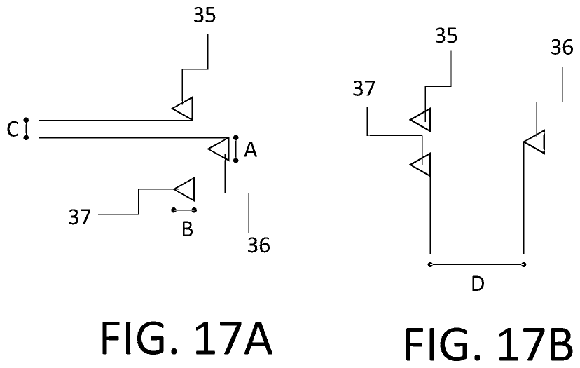

Generally, and referring now specifically to FIGS. 16A-16C and 17A-17B, the plurality of through openings 35, 36, 37, 38, 39 are arranged perpendicular to a longitudinal axis L of the outlet pipe 4, particularly with a main axis X arranged extending perpendicular to a longitudinal axis L of the outlet pipe 4. The plurality of through openings 35, 36, 37, 38, 39 each comprise a shape including a tip end 40 (cf. FIGS. 16A-16C) pointing in an upstream direction U, i.e. in a direction towards the water seal 6 of the toilet system 1, or in a direction opposite to the direction of flow 34.

The plurality of through openings 35, 36, 37, 38, 39 each comprise a size A (cf. FIG. 17A) measured in a direction perpendicular to the longitudinal axis L of the outlet and a size B (cf. FIG. 17A) measured in a direction parallel with the longitudinal axis L of the outlet. Neighboring through openings are arranged spaced apart with a distance C (cf. FIG. 17A) measured in a direction perpendicular to the longitudinal axis L of the outlet and a distance D (cf. FIG. 17B) measured in a direction parallel with the longitudinal axis L of the outlet. The plurality of openings 35, 36, 37, 38, 39 are arranged in a pattern fulfilling the relations C<A and D<2B.

The plurality of through openings 35, 36, 37, 38, 39 may comprise a cross sectional shape being triangular (cf. FIG. 16B), oval (cf. FIG. 16A), diamond-shaped or any combination thereof. In case of a triangular opening, the main axis X corresponds to a base of the triangle. In case of an oval or ellipsoid opening, the main axis X corresponds to a minor axis of the oval or ellipsoid. FIG. 16C illustrate another example of an opening 35 being triangular with a straight base and concave sides. The plurality of through openings 35, 36, 37, 38, 39 may further be dimensioned such that the size A is between 0.5 mm and 100 mm and/or such that the size B is between 0.5 mm and 100 mm.

Generally, any number of through openings 35, 36, 37, 38, 39 may be provided. In particular, the number of through openings 35, 36, 37, 38, 39 is five or more, such as six or eight.

The sensor device 10 may further comprise a screening device 52. The screening device 52 may as shown on FIG. 14 be arranged extending over the through openings 35, 36, 37, 38, 39, e.g. as a grating with holes sized to allow liquid, but not solids particles above a predetermined size to pass. Alternatively, the screening device 52 may as shown on FIG. 15 be arranged in a position upstream of the through openings 35, 36, 37, 38, 39. The screening device 52 diverts solids such as feces to flow around the through openings 35, 36, 37, 38, 39 and thereby serves to hinder solids such as feces in falling into the through openings 35, 36, 37, 38, 39 and potentially blocking them. The screening device 52 is furthermore constructed to allow liquids, such as water, to flow around the screening device 52 and fall through the through openings 35, 36, 37, 38, 39 into the chamber 11, while still allowing solids, such as feces, to be led past the through openings 35, 36, 37, 38, 39.

The sensor device 10, or at least the chamber 11 of the sensor device 10, may be configured to be attached to, such as in one piece with, the outlet pipe 4 of the toilet system 1. The sensor device 10, or at least the chamber 11 of the sensor device 10, may also be configured as a pipe section to be attached to and form an integral part of the outlet pipe 4 of the toilet system 1. The plurality of openings 35, 36, 37, 38, 39 may be provided in the existing outlet pipe 4 of the toilet system 1. The plurality of openings 35, 36, 37, 38, 39 may also be provided in the pipe section to be attached to the existing outlet pipe 4 of the toilet system 1.

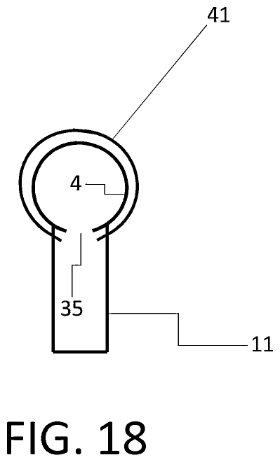

Referring now also to FIG. 18, the sensor device 10 may further comprise a mounting element 41 adapted for enabling mounting the chamber 11 of the sensor device 10 on the outlet pipe 4 of the toilet system 1.

The mounting element 41 may be a welding, a gluing or the like as indicated on FIG. 14. The mounting element 41 may also generally comprise a semi-ring-shaped element configured to extend around and be attached to and/or tightened to the outlet pipe 4 as shown on FIG. 18.

The mounting element 41 may also be a flexible element being integrally connected to the chamber 11 with at least a first end and being adapted for being wrapped around the outlet pipe 4 and attached with a second end to the chamber 11, for instance in a manner like a terminal strip. In such an embodiment, the connection between the second end of the mounting element 41 and the chamber 11 may for instance be a snap-locking or friction-locking connection.

Referring again to FIG. 1, the sensor device 10 may further comprise a measuring unit 14 configured to measure the quantity of liquid 13 collected in the chamber 11. The sensor device 10 may further comprise an outlet 12 arranged in a bottom area of the chamber 11 and connected to a sewer system or the like. The sensor device 10 may still further comprise a closure mechanism 32, such as a valve, a flap or a shutter, configured to be closed while the measuring unit 14 measures the quantity of liquid 13 collected in the chamber 11 and to be opened subsequently to allow the chamber 11 to be emptied and the toilet system 1 to be flushed when the toilet system 1 is flushed.

Referring now to FIG. 19, a schematic cross-sectional side view similar to those of FIGS. 14 and 15 and illustrating a toilet system according to the invention with an alternative embodiment of the sensor device 10 is shown. The embodiment shown on FIG. 19 differs from those of FIGS. 14 and 15 in virtue of the following features.

The sensor device 10 comprises a pump 55 for evacuating the contents of the chamber 11 back into the outlet pipe 4. The pump 55 may be mounted inside the chamber 11 or outside the chamber and connected to the chamber 11. The pump 55 may be connected to a pipe or hose 56, which in turn may be connected to an inlet 57 leading to the outlet 4 of the toilet system 1 at a position downstream of the at least one opening 35.

The sensor device 10 comprises in this embodiment only one opening 35 provided in the outlet 4 of the toilet system. At or in connection with the opening 35 is provided a valve 58. The valve 58 is provided on the side of the opening 35 facing the chamber 11, and thus outside of the outlet 4. The valve 58 may be of a standard type or it may be custom made. The valve 58 comprises an actuator 59 and an outlet 61. The outlet 61 opens or debouches into the chamber 11, such that liquid lead through the opening 35 is lead through the valve 58 and its outlet 61 into the chamber 11 (flow 51). The actuator 59 may for instance be a motion sensor or a contact sensor which actuates the valve, e.g. upon registering the motion or contact of a liquid flow. The valve 58 may be configured to have a filtering tolerance such as to only let through particles of a suitably small size, such as below 10 mm or below 5 mm or below 0.5 mm. The valve 58 may further be configured to have a maximum capacity enabling the valve 58 to handle a predetermined maximum flow or volume of liquid, for instance corresponding to an expected or statistically feasible maximum volume of an excretion.

It is noted that in embodiments where the sensor device 10 comprises more than one opening 35-39, each such opening 35-39 may comprise an associated valve 58 as described above. However, embodiments of the sensor device 10 comprising a valve 58 generally comprises only one opening 35.

The sensor device 10 according to FIG. 19 further comprises a data processing device 61. The data processing device 61 may have one or more different functions. The data processing device 61 may transmit measurement data detected by the sensor device 53, 54 to a data analysis device. The data processing device 61 may control the actuator 59 of the valve 58. The data processing device 61 may control the pump 55. It is noted that such a data processing device 61 may also optionally be provided to any of the sensor devices 10 described further above, e.g. with respect to FIGS. 14 and 15.

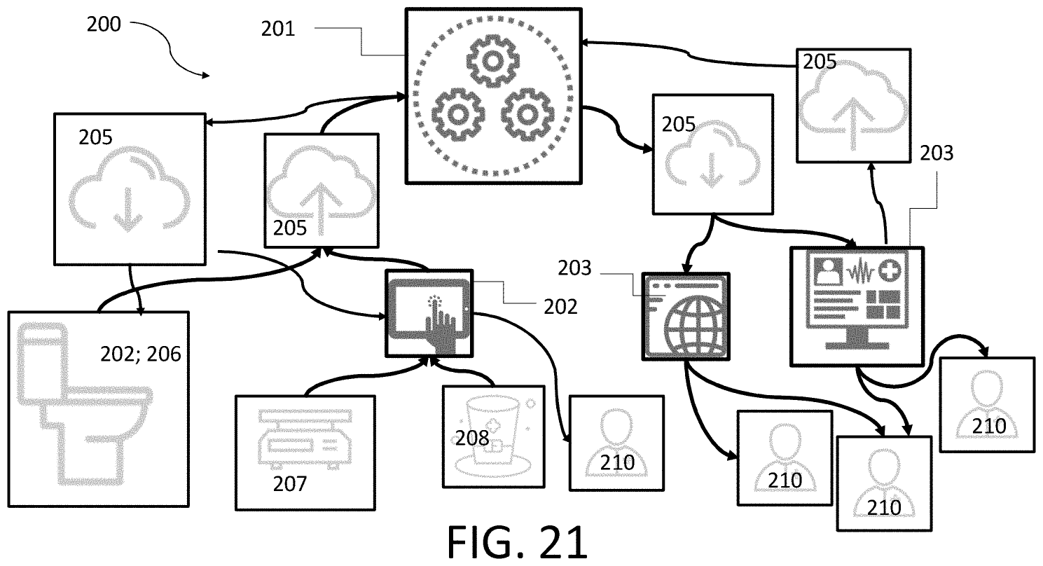

Referring now to FIG. 21, a first embodiment of a fluid balance monitoring system 200 for determining and monitoring the fluid balance of a mammalian subject 100 (FIG. 20) according to the invention is shown.

The fluid balance monitoring system 200 according to the invention generally comprises a data processing unit 201 configured to manage and process measurement data, one or more input units 202 configured to receive measurement data and transmit the received measurement data to the data processing unit 201, and one or more display units 203 configured to receive output data indicative of the monitored fluid balance of the subject 100 and to display the output data indicative of the monitored fluid balance of the subject 100.

The one or more input units 202 are configured to receive measurement data and transmit the received measurement data to the data processing unit 201. The one or more input units 202 are in data transferring connection with the data processing unit 201. The one or more input units 202 may comprise a data processing unit. The one or more input units 202 may comprise a display. Generally, the data processing unit 201, the one or more input units 202 and the one or more display units 203 are mutually, particularly physically, separate units.

Generally, subjects 100, particularly humans, tend to move around and may thus experience inflow 101 and outflow 102 of liquids at different locations. For instance, subjects 100 generally defecate at different physical locations than where they eat. Thus, for easy registration of inflow 101 and outflow 102 of liquid, it is needed to enable recording of measurement data at different locations. The one or more input units 202 are therefore configured to enable collection of data at several different locations. Suitable input units 202 thus include a mobile telephone, a tablet computer and a laptop computer as well as other similar portable devices. The one or more input units 202 may receive measurement data by means of a user interface. Alternatively, or additionally, the one or more input units 202 may be configured to receive measurement data from sensors or other relevant devices, such as the devices 206, 207 and 208 shown in FIG. 21.

For instance, a toilet system 206 with sensors or an electronic scale 207 may be suitable for providing data indicative of fluid outflow 102. An electronic scale 207 may also provide data indicative of the weight and change in weight of the subject 100. Data indicative of fluid inflow 101 may for instance be measured by using, e.g., plates, glasses or cups 208 with built-in sensors of a suitable type, such as volume or weight sensors. Devices such as toilet systems 206, scales 207 and cups 208 as described above may also function as an input unit 202 in itself, for instance if being enabled for Internet of Things (IoT) or if comprising a data processing device or a data transmitter. Furthermore, it is feasible to preprogram software of the one or more input units 202 to enable display on a screen of the input unit 202, e.g. a touchscreen, of preset types and volumes, such that it is only time that is a novel variable.

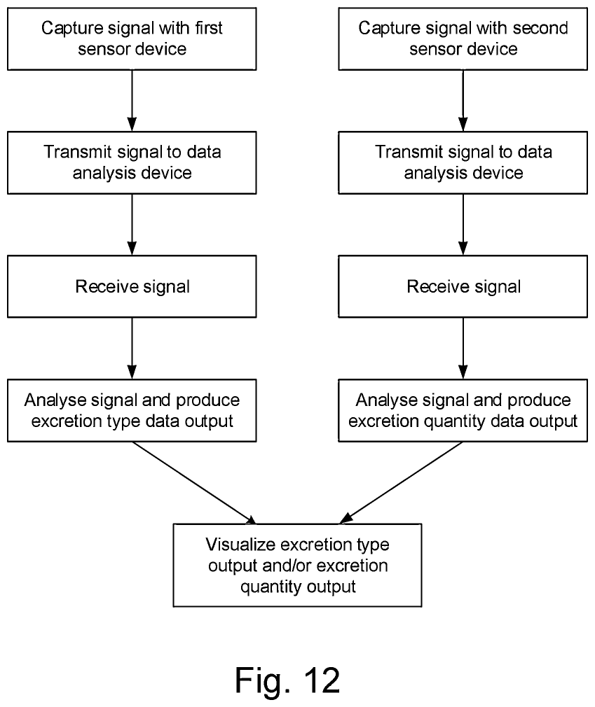

It is noted in this connection that suitable toilet systems 206 with sensors are known in the art and include a toilet system as that described in the applicant's Danish patent application no DK PA 2020 70063 and/or in the above in relation to FIGS. 1-19, and/or as that described in US 2018/368818 A1.

Generally, the measurement data may comprise any data or information relevant for monitoring the fluid balance of a subject 100. The measurement data may comprise information regarding quantity, type and time for each event and individual subject 100. The information regarding quantity, type and time for each event may be saved together with or encrypted by an individual identification key to allow further summation for a specific individual.

An event may in this connection be any relevant event leading to an inflow 101 or an outflow 102 of fluid from a subject 100. Non-limiting examples include excretion, such as defecation and urination, output from drain intake of food or drink, exercise, perspiration, respiration, sputum secretion, mucosal secretion or gastric evacuation. The measurement data may also comprise data such as weight data regarding the subject 100.

The input unit 202 may further be configured to upload the measurement data to a cloud-based storage 205 or directly to a storage of the data processing unit 201. The input unit 202 may further be configured to provide the measurement data with an identification key for identifying the relevant subject 100 related to the measurements. The input unit 202 may still further be configured to receive data, for instance a request, from the data processing unit 201 via a cloud-based storage 205 or directly from the data processing unit 201. The request may be a request for providing new or further measurement data. The request may furthermore specify the measurement data required.

The data processing unit 201 is a central data processing unit in the sense that the fluid balance monitoring system 200 is configured such that almost all or all data communication between the one or more input units 202 and the one or more display units 203 is lead through the data processing unit 201. The data processing unit 201, the one or more input units 202 and the one or more display units 203 are thus mutually separate units. The fluid balance monitoring system 200 according to the invention is thus centrally managed.

The data processing unit 201 is configured to communicate with the one or more input units 202 and the one or more display units 203. The data processing unit 201 is configured to receive measurement data from the one or more input units 202 and to process the received measurement data to obtain output data indicative of the of the monitored fluid balance of the subject 100. The obtained output data indicative of the of the monitored fluid balance of the subject 100 may be a summarization of the data, e.g., in one or more tables or one or more graphs. The obtained output data may also be in any other format suitable for being displayed by the one or more display devices 203. The data processing unit 201 is further configured to transmit the obtained output data indicative of the of the monitored fluid balance of the subject 100 to the one or more display units 203. The data processing unit 201 is thus connected in a data transferring relationship with the one or more input units 202 and the one or more display units 203, where the connection may be wireless or wired, such as via a local area network (LAN) a wide area network (WAN) or the Internet.

The data processing unit 201 may still further be configured to transmit data, for instance a request, to the input unit 202 via a cloud-based storage 205 or directly to the input unit 202. The request may be a request for providing new or further measurement data. The request may furthermore specify the measurement data required. The request may be formed based on data, such as a request, received from the one or more display units 203. Alternatively, or additionally, the request may be sent provided no measurement data has been received at the data processing unit 201 for a predetermined amount of time.

The data transfer or transmission between the data processing unit 201 and the one or more input units 202 and one or more display units 203, respectively, may be direct or may be via intermediate storage, e.g. in a cloud-based storage 205. The data processing unit 201 may also comprise an internal storage. Data stored in a cloud-based storage 205 or in the internal storage of the data processing unit 201 may be associated with, e.g. stored together with or encrypted by, an individual identification key. The identification key may provide or comprise a suitable identification of a subject 100. The identification of the subject 100 may be anonymous such as to conform with relevant regulations, particularly data protection regulations such as the GDPR regulations in force within the European Union.

The one or more display units 203 are configured to receive the obtained output data indicative of the of the monitored fluid balance of the subject 100 from the data processing unit 201. The one or more display units 203 are in data transferring connection with the data processing unit 201. The one or more display units 203 are configured to display the obtained output data indicative of the of the monitored fluid balance of the subject 100. The one or more display units 203 thus comprise a display. More particularly, the one or more display units 203 are configured to display the obtained output data indicative of the of the monitored fluid balance of the subject 100 in a format suitable for easy and straight forward interpretation by a viewer 210. Such a format may for example include graph(s) or table(s). Typically, the viewer 210 is a health professional, such as a nurse or a doctor. As health professional are frequently on the move during their daily routines, suitable display units 203 include a mobile telephone, a tablet computer and a laptop computer as well as other similar portable devices. The one or more display units 203 may comprise a data processing unit, for instance to enable a final processing of the output data to allow displaying it. The one or more display units 203 may therefore in principle be any display unit comprising a processing unit connected to the data processing unit 201.

The one or more display units 203 may still further be configured to transmit data, for instance a request, to the data processing unit 201 via a cloud-based storage 205 or directly to the data processing unit 201. The request may be a request for providing new or further measurement data. The request may furthermore specify the measurement data required.

Since the measurement data are delivered to a central data processing unit 201 and processed to output data therein, processed measurement data originating from any input unit 202 may be displayed on any given display unit 203. The output data obtained by the data processing unit 201 and indicative of the of the monitored fluid balance of the subject 100 may thus be viewed on the display unit 203. The obtained output data may comprise any desired information. Examples are summarized data, like an overview of the fluid balance of the subject 100, total inflow 101 and outflow 102, and the contribution of each measuring unit or measurement to the total fluid balance. Likewise, the fluid balance of the subject 100 for a given period can be displayed on the display unit 203. The display unit 203 may further be configured to update the displayed data continuously or with a determined time interval.

In the above description, the display unit 203, the input unit 202 and the data processing unit 201 are envisaged to be different physical units. It is noted, however, that the display unit 203 and the input unit 202 may also be the same physical unit. It is even possible that the display unit 203, the input unit 202 and the data processing unit 201 may be one and the same physical unit. In both cases the units may still be set up to ensure that all data communication goes through the data processing unit 201. Irrespective of the embodiment, the display unit 203 and the input unit 201 are synchronized.

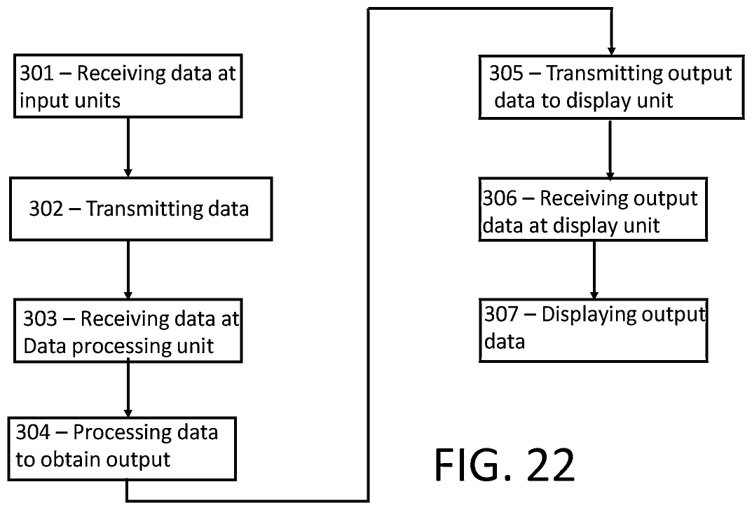

Turning now to FIG. 22, a method for determining and monitoring the fluid balance of a mammalian subject 100 according to the invention will be described. The method comprises the following steps.

First, a fluid balance monitoring system 200 according to the invention is provided. The fluid balance monitoring system 200 comprises at least one data processing unit 201 with at least one data processing device, at least one input unit 202 and at least one display unit 203 with at least one data processing device.

In step 301, the at least one input unit 202 is used to receive, from any one or more of a user interface and at least one sensor device 206, 207, 208, measurement data comprising information relevant for determining and monitoring the fluid balance of a subject 100. The measurement data comprises information regarding a quantity, a type and a time of occurrence for at least one event relevant for determining and monitoring the fluid balance of a subject 100, where the at least one event is any one relevant event leading to an outflow of fluid from the subject or an inflow of fluid to the subject 100.

In step 302, the at least one input unit 202 is used to transmit the received measurement data to the at least one data processing unit 201.

In step 303, the at least one data processing unit 201 receives the measurement data from the at least one input unit 202.

In step 304, the at least one data processing unit 201 is used to process the received measurement data to achieve output data indicative of the fluid balance of a subject 100.

In step 305, the at least one data processing unit 201 transmits the output data indicative of the fluid balance of a subject 100 to the at least one display unit 203.

In step 306, the at least one display unit 203 receives the output data indicative of the fluid balance of a subject 100.

Finally, in step 307, the at least one display unit 203 displays the output data indicative of the fluid balance of a subject 100 and further indicative of a type and a time of occurrence of the at least one event.

In a further, optional, step data, such as measurement data or the output data indicative of the fluid balance of a subject 100, may be uploaded to or saved in the subject's case sheet.

The person skilled in the art realizes that the present invention by no means is limited to the embodiments described above. On the contrary, many modifications and variations are possible within the scope of the appended claims.

What is claimed is:

1. A toilet system comprising a bowl and an outlet, the toilet system being adapted for discerning between types of excretion excreted into the bowl by a user of the toilet system, the toilet system further comprising:
at least one first sensor device configured to capture at least one signal indicative of a type of excretion excreted into the bowl by a user of the toilet system, and
at least one second sensor device configured to capture at least one signal indicative of a quantity of excretion excreted into the bowl by a user of the toilet system.

2. A toilet system according to claim 1, wherein the at least one first sensor device is configured to measure sounds, and/or
wherein the at least one first sensor device comprises an acoustic sensor or a microphone or a vibration sensor.

3. A toilet system according to claim 1, wherein the at least one first sensor device is arranged in or at any one of the toilet seat, the bowl above a waterline and the bowl below the waterline.

4. A toilet system according to claim 1, wherein the at least one first sensor device further comprises any one or more of a pressure sensor, a radar, an image capturing device, a capacitive sensor, and flow rate sensor, and/or
wherein the at least one first sensor device is further configured to transmit the at least one signal indicative of a type of excretion excreted into the bowl by a user of the toilet system to a data analysis device.

5. A toilet system according to claim 1, wherein the at least one second sensor device is configured to measure a volume of liquid indicative of a quantity of excretion excreted into the bowl by a user of the toilet system, and/or
wherein the at least one second sensor device is arranged in the outlet of the toilet system in a position downstream of a water seal arranged between the bowl and the outlet of the toilet, and/or
wherein the at least one second sensor device is further configured to transmit the at least one signal indicative of a quantity of excretion excreted into the bowl by a user of the toilet system to a data analysis device.

6. A toilet system according to claim 1, and further comprising a data analysis device, the data analysis device comprising a data processing unit and a data storage unit, the data processing unit being configured to:
receive one or more signals transmitted by the at least one first sensor device, and
analyse the received one or more signals such as to produce a data output indicative of at least the type of excretion excreted into the bowl by a user of the toilet system.

7. A toilet system according to claim 6, wherein the data analysis device is further configured to:
receive one or more signals transmitted by the at least one second sensor device, and
analyse, using the data processing unit, the received one or more signals such as to produce a data output indicative of the quantity of excretion and the quantity of excretion excreted into the bowl by a user of the toilet system,
and/or
wherein the data analysis device further comprises a data visualization unit, and the data processing unit is further configured to visualize on the data visualization unit the data output indicative of one or more of the type of excretion excreted into the bowl by a user of the toilet system and the quantity of excretion excreted into the bowl by a user of the toilet system.

8. A toilet system according to claim 1, and further comprising any one or more of:
an actuator configured to allow a user to indicate a type of excretion excreted into the bowl by the user of the toilet system and to transmit a signal indicative of the user's indication to an analysis device,
an actuator configured to allow a user to activate the toilet system and/or the analysis device before use of the toilet system, and
a weight sensor arranged and configured to capture signals indicative of the user's weight before and after excretion, respectively.

9. A toilet system according to claim 1, wherein the first sensor device is an image capturing device arranged in or at any one of the toilet seat and the bowl above a waterline, and wherein
the second sensor device is arranged in the outlet of the toilet system in a position downstream of a water seal arranged between the bowl and the outlet of the toilet and comprises a chamber adapted for collecting liquid forced through the water seal and a weight sensor arranged and adapted for measuring the weight of the chamber and liquid contained in the chamber.

10. A toilet system according to claim 9, wherein the first sensor device is arranged such as to point downwards at an angle (α) of between 70 and 80 degrees, such as an angle (α) of 75 degrees, with the horizontal (H) or in an angle (β) of between 20 and 10 degrees, such as an angle (β) of 15 degrees, with the vertical (V), where an angle of 90 degrees in this context corresponds to the vertical (V) or the direction of gravity, and/or wherein
the first sensor device is located outside the central axis (A) of the seat and is rotated around the direction of gravity or vertical (V) to point in an angle (γ) of between 25 and 25 degrees, such as an angle (γ) of 30 degrees, with the central axis (A) of the seat.

11. A method for determining a type of excretion excreted into a bowl of a toilet system according to claim 1 by a user of the toilet system, the method comprising the steps of:
using at least one first sensor device of the toilet system, capturing a signal indicative of a type of excretion excreted into the bowl by a user of the toilet system,
transmitting the captured signal indicative of a type of excretion excreted into the bowl by a user of the toilet system to an analysis device comprising a data processing unit and a data storage unit,
receiving, by means of the data processing unit, one or more signals transmitted by the at least one first sensor device, and
analysing, by means of the data processing unit, the received one or more signals transmitted by the at least one the first sensor device such as to produce a data output indicative of the type of excretion excreted into the bowl by a user of the toilet system.

12. A method according to claim 11, and comprising the further steps of:
using at least one second sensor device of the toilet system, capturing a signal indicative of a quantity of excretion excreted into the bowl by a user of the toilet system,
transmitting the captured signal indicative of a quantity of excretion excreted into the bowl by a user of the toilet system to the analysis device,
receiving, by means of the data processing unit, one or more signals transmitted by the at least one the second sensor device, and
analysing, by means of the data processing unit, the received one or more signals transmitted by the at least one the second sensor device such as to produce a data output indicative of the quantity of excretion excreted into the bowl by a user of the toilet system,
and/or
wherein the data analysis device further comprises a data visualization unit, and the method further comprises the step of:
visualizing on the data visualization unit, using the data processing unit, the data output indicative of the type of excretion excreted into the bowl by a user of the toilet system and/or the data output indicative of the quantity of excretion excreted into the bowl by a user of the toilet system.

13. A toilet system comprising a bowl and an outlet the toilet system being adapted for discerning between at least types of excretion excreted into the bowl by a user of the toilet system, the toilet system further comprising at least one sensor device configured to capture at least one signal indicative of a quantity of excretion excreted into the bowl by a user of the toilet system, the sensor device comprising:
a chamber provided on the outlet of the toilet system in a position downstream of a water seal arranged between the bowl and the outlet of the toilet system, the chamber being arranged adjacent to at least a part of a lower half of the outlet pipe of the toilet system and being adapted to receive a quantity of liquid pressed through a water seal of the toilet system, and
at least one through opening provided in the outlet of the toilet system in the position downstream of the water seal at which the chamber is provided such as to allow liquid being forced through the water seal to flow into the chamber, wherein
the at least one through opening is arranged with a main axis (X) extending perpendicular to a longitudinal axis (L) of the outlet pipe, and wherein
the at least one through opening comprises a shape tapering in an upstream direction.

14. A toilet system according to claim 13, wherein a plurality of through openings is provided in the outlet of the toilet system,
wherein the plurality of through openings comprise a largest size A measured in a direction perpendicular to the longitudinal axis of the outlet pipe and a largest size B measured in a direction parallel with the longitudinal axis of the outlet pipe,
wherein neighboring through openings of the plurality of through openings are arranged spaced apart with a shortest distance C measured in a direction perpendicular to the longitudinal axis of the outlet pipe and a shortest distance D measured in a direction parallel with the longitudinal axis of the outlet pipe, and
wherein the plurality of through openings are arranged in a pattern fulfilling the relations C<A and D<2B.

15. A toilet system according to claim 13, wherein the at least one through opening a shape tapering and ending in a tip end pointing in an upstream direction, and/or
wherein the at least one through opening comprises a cross sectional shape being polygonal, ellipsoid, circular, with any one or more of regular, concave, convex or irregular edge(s), or any combination thereof.

16. A toilet system according to claim 13, wherein the at least one through opening comprises a largest size A measured in a direction perpendicular to the longitudinal axis of the outlet pipe and a largest size B measured in a direction parallel with the longitudinal axis of the outlet pipe, and
wherein the size A is between 0.5 mm and 100 mm, and/or
wherein the size B is between 0.5 mm and 100 mm.

17. A toilet system according to claim 14, wherein the plurality of through openings comprise at least five openings.

18. A toilet system according to claim 13, wherein the sensor device further comprises any one or more of:
a screening device arranged upstream of the at least one through opening or extending over the at least one through opening, and
a valve mounted in connection with at least one of the one or more openings and configured to control inflow of liquid into the chamber.

19. A toilet system according to claim 13, wherein the sensor device further comprises:
a mass sensor or a weight sensor arranged and configured to monitor a weight of the chamber and to capture at least one signal indicative of a quantity of excretion excreted into the bowl by a user of the toilet system, and/or
a flow sensor arranged and configured to monitor a flow of liquid through the chamber and to capture at least one signal indicative of a quantity of excretion excreted into the bowl by a user of the toilet system.

20. A toilet system according to claim 13, wherein the sensor device further comprises a mounting element adapted for enabling mounting the sensor device on an outlet pipe of a toilet system.

21. A method of providing a toilet system according to claim 13, with at least one sensor device configured to capture at least one signal indicative of a quantity of excretion excreted into the bowl by a user of the toilet system, the method further comprising the steps of:
providing at least one through opening in at least a part of a lower half of the outlet of the toilet system in a position downstream of a water seal arranged between the bowl and the outlet of the toilet system such as to allow liquid being forced through the water seal to flow through the at least one through opening and into the chamber, wherein
the at least one through opening is arranged with a main axis extending perpendicular to a longitudinal axis L of the outlet pipe, and wherein
the at least one through opening comprises a shape including a tip end pointing in an upstream direction,
providing a chamber adapted to receive a quantity of liquid pressed through a water seal of the toilet system, and
arranging the chamber on the outlet of the toilet system in the position downstream of the water seal arranged between the bowl and the outlet of the toilet system and adjacent to at least a part of a lower half of the outlet pipe of the toilet system.

22. A method according to claim 21, and comprising one or more of the further steps of:
providing a plurality of through openings to comprise a size A measured in a direction perpendicular to the longitudinal axis L of the outlet and a size B measured in a direction parallel with the longitudinal axis L of the outlet, wherein neighboring through openings are arranged spaced apart with a distance C measured in a direction perpendicular to the longitudinal axis L of the outlet and a distance D measured in a direction parallel with the longitudinal axis L of the outlet, and wherein the plurality of openings are arranged in a pattern fulfilling the relations C<A and D<2B,
providing the at least one through opening or the plurality of through openings to comprise a cross sectional shape being polygonal, ellipsoid, circular, with any one or more of regular, concave, convex or irregular edge(s), or any combination thereof,
providing the at least one through opening or the plurality of through openings to comprise a largest size A measured in a direction perpendicular to the longitudinal axis L of the outlet and a largest size B measured in a direction parallel with the longitudinal axis L of the outlet, where the size A is between 0.5 mm and 100 mm, and alternatively, or additionally, where the size B is between 0.5 mm and 100 mm, and
providing the plurality of through openings to comprise at least five openings.

23. A fluid balance monitoring system adapted for determining and monitoring the fluid balance of a mammalian subject, the fluid balance monitoring system comprising at least one data processing unit, at least one input unit and at least one display unit,
wherein the at least one input unit is configured to:
receive, from any one or more of a user interface and at least one sensor device, measurement data comprising information relevant for determining and monitoring the fluid balance of a mammalian subject, and
transmit the received measurement to the at least one data processing unit,
wherein the at least one data processing unit comprises a data processing device, and the at least one data processing unit is configured to:
receive the measurement data from the at least one input unit,
process the received measurement data to achieve output data indicative of the fluid balance of the mammalian subject, and
transmit the output data indicative of the fluid balance of a subject to the at least one display unit,
wherein the at least one display unit comprises a data processing device and the at least one display unit is configured to:
receive the output data indicative of the fluid balance of the mammalian subject, and
display the output data indicative of the fluid balance of the mammalian subject,
wherein the measurement data further comprises information regarding a quantity, a type and a time of occurrence for at least one event relevant for determining and monitoring the fluid balance of the mammalian subject, where the at least one event is any one relevant event leading to an outflow of fluid from the mammalian subject or an inflow of fluid to the mammalian subject, and
wherein the at least one data processing unit furthermore is configured to process the received measurement data to achieve output data which is further indicative of a type and a time of occurrence of the at least one event.

24. A fluid balance monitoring system according to claim 23, wherein the at least one data processing unit is in data transferring connection with the at least one input unit and the at least one display unit in such a way that all data communication between the at least one input unit and the at least one display unit is lead through the at least one data processing unit.

25. A fluid balance monitoring system according to claim 23, wherein the measurement data comprises data related to at least one event relevant for determining and monitoring the fluid balance of the mammalian subject, where the at least one event includes any one or more of: excretion, defecation, urination, intake of food or drink, intravenous treatment, subcutaneous treatment, exercise, output from drain, output from ulcers, perspiration, respiration, sputum secretion, mucosal secretion, and gastric evacuation.

26. A fluid balance monitoring system according to claim 23, wherein at least one of the measurement data and the output data is saved in a cloud-based storage or a storage provided in the data processing unit, preferably together with or encrypted by an individual identification key.

27. A fluid balance monitoring system according to claim 23, wherein the output data comprises at least data indicative of an inflow of liquid to the mammalian subject, an outflow of liquid from the mammalian subject and a change in the fluid balance of the mammalian subject.

28. A fluid balance monitoring system according to claim 26, wherein the display unit is configured to display the output data in a manner showing the data indicative of an inflow of liquid to the mammalian subject, an outflow of liquid from the mammalian subject and a change in the fluid balance of the mammalian subject separately from one another.

29. A fluid balance monitoring system according to claim 23, wherein the data processing unit is configured to generate output data and send the output data to the at least one display unit in real time or with predetermined time intervals, and/or wherein the display unit is configured to display the output data in real time or with predetermined time intervals.

30. A method for determining and monitoring the fluid balance of a mammalian subject, the method comprising the steps of:
a) providing a fluid balance monitoring system according to claim 23 and comprising at least one data processing unit with at least one data processing device, at least one input unit and at least one display unit with at least one data processing device,
b) receiving, with the at least one input unit and from any one or more of a user interface and at least one sensor device, measurement data comprising information relevant for determining and monitoring the fluid balance of a mammalian subject, and
c) transmitting, with the at least one input unit, the received the measurement data to the at least one data processing unit,
d) receiving, with the at least one data processing unit, the measurement data from the at least one input unit,
e) processing, with the at least one data processing unit, the received measurement data to achieve output data indicative of the fluid balance of the mammalian subject,
f) transmitting, with the at least one data processing unit, the output data indicative of the fluid balance of the mammalian subject to the at least one display unit,
g) receiving, with the at least one display unit, the output data indicative of the fluid balance of the mammalian subject, and
h) displaying, with the at least one display unit, the output data indicative of the fluid balance of the mammalian subject,
wherein the measurement data further comprises information regarding a quantity, a type and a time of occurrence for at least one event relevant for determining and monitoring the fluid balance of the mammalian subject, where the at least one event is any one relevant event leading to an outflow of fluid from the mammalian subject or an inflow of fluid to the mammalian subject, and
comprising the further step of processing, with the at least one data processing unit, the received measurement data to achieve output data which is further indicative of a type and a time of occurrence of the at least one event.

31. A method according to claim 30, wherein:
the at least one data processing unit is in data transferring connection with the at least one input unit and the at least one display unit in such a way that all data communication between the at least one input unit and the at least one display unit is lead through the at least one data processing unit, or
wherein the first measurement data and second measurement data comprises data related to at least one event relevant for determining and monitoring the fluid balance of the mammalian subject, where the at least one event includes any one or more of: excretion, defecation, urination, intake of food or drink, exercise, perspiration and respiration, or
wherein the output data comprises at least data indicative of an inflow of liquid to the mammalian subject, an outflow of liquid from the mammalian subject and a change in the fluid balance of the mammalian subject.

32. A method according to claim 30, and comprising one or more of the further steps of:
saving at least one of the measurement data and the output data in a cloud-based storage or a storage provided in the data processing unit, preferably together with or encrypted by an individual identification key,
generating, with the data processing unit, output data in real time and sending, with the data processing unit, the output data to the at least one display unit in real time or with predetermined time intervals, and
displaying, with the display unit, the output data in real time or with predetermined time intervals.
